(19) United States
(12) Patent Application Publication
(10) Pub. No.: US 2002/0164651 A1
(43) Pub. Date: Nov. 7, 2002

(54) Diagnostic for inflammatory diseases associated with cartilage and bone surfaces

(76) Inventor: Marla Steinbeck, Doylestown, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 10/073,402

(22) Filed: Feb. 11, 2002

Related U.S. Application Data
(60) Provisional application No. 60/268,847, filed on Feb. 15, 2001.

(51) Int. Cl. G01N 33/53; C12P 19/00; G01N 33/543

(52) U.S. Cl. 435/7.1; 436/518

(57) ABSTRACT

The presence of activated neutrophils in areas of inflammation in arthritic joints and in periodontitis results in the deposition of myeloperoxidase onto the extracellular matrix protein surface. The resultant production of the reactive HOCl, Cl₂, and N-chloramines causes the pyridinium cross-links and lysine/hydroxylysine amino acids in the collagen to be oxidized, thereby leading to an increased susceptibility of the collagen to proteolytic degradation, with the subsequent loss of matrix proteins at sites of inflammation. The present invention relates to methods of diagnosing inflammatory diseases or conditions by detecting and monitoring the level of chlorinated compound(s) that result from the oxidation of pyridinum and and lysine/hydroxylysine amino acids of collagen types I, II, III, IX, and XI by HOCl, Cl₂, and N-chloramines.

### CROSS REFERENCE TO RELATED APPLICATIONS

This application claims priority, in part, under U.S.C. 35 §119 based upon U.S. Provisional Application No. 60/268,847 filed Feb. 15, 2001.

### GOVERNMENT RIGHTS TO THE INVENTION

The invention was made with government support under grants DE-08507 and DE-11082 awarded by the National Institute of Health. The government has certain rights to the invention.

### FIELD OF THE INVENTION

The present invention relates to the fields of immunology and matrix biology, and to a method of diagnosing, and monitoring the progression of, the damage/loss of matrix proteins at sites of inflammation and more particularly, to the oxidation/chlorination of pyridinoline/pyrole/ketoimine and lysine/hydroxlylysine residues cross-links in collagen types I, II, III, IX, and XI by reactive oxygen species.

### BACKGROUND OF THE INVENTION

Inflammation associated with articular cartilage, bone, and dentin surfaces is characterized by accumulation, adhesion, and activation of neutrophils and monocytes, which results in the destruction of cartilage and the loss of bone at these sites. (Woolley, D. et al..,, Ann Rheum Dis. 56(3), 151-61, 1997; Fredriksson, M., et al.., J Clin Periodontol 25(5), 394-8, 1998; Fredriksson, M. I., et al.., J Periodontal 70(11), 1355-1360, 1999; Harris, E. D. J. Inflammation: Basic principles and Clinical Correlates (Gallin, J. I., Goldstein, I. M., Snyderman, R., ed), 751-774, Raven Press, Ltd., New York, 1988) These effects are thought to be mediated in part by the production of reactive oxygen species (ROS), a group of reactants that includes superoxide (O₂), hydrogen peroxide (H₂O₂), and the highly reactive species hypochlorous acid (HOCl) and singlet oxygen (O₂(Δg)). (Babior, B. M. Blood 64(5), 959-66, 1984; Klebanoff, S. J. Inflammation: Basic Principles and Clincal Correlates (Gallin, J. I., Goldstein, I. M., Snyderman, R., ed), 391-444, Raven Press, Ltd., New York, 1988; Halliwell, B. Ann Rheum Dis 54(6), 505-10, 1995; Greenwald, R. A. Semin Arthritis Rheum 20(4), 219-40, 1991) ROS are produced by neutrophils and monocytes after they are recruited from the circulation to extravascular spaces. Once outside of the circulation, they adhere to extracellular matrix proteins (ECM) and undergo activation, which results in the production of ROS and release of proteolytic enzymes directly onto the matrix surface. (Harris, E. D. J. Inflammation: Basic principles and Clinical Correlates (Gallin, J. I., Goldstein, I. M., Snyderman, R., ed), 751-774, Raven Press, Ltd., New York, 1988; Ginis, I., Tauber, A. I., Blood 76 (6), 1233-9, 1990; Weiss, S. J., LoBuglio, A. F., Lab Invest 47(1), 5-18, 1982; Harlan, J. M. Blood 65(3), 513-25 1985; Vissers, M. C., et al.., Blood 66(1), 161-6, 1985; Henson, P. M., Johnston, R. B., Jr. J Clin Invest 79(3), 669-74, 1987) In inflammation associated with arthritic joints, the accumulation and activation of neutrophils and monocytes and increased synovial cell formation result in the loss of synovial membrane integrity and eventually to irreversible damage and destruction of articular cartilage in the afflicted joints. (Woolley, D. E., et al.., Ann Rheum Dis. 56(3), 151-61, 1997; Harris, E. D. J. Inflammation: Basic principles and Clinical Correlates (Gallin, J. I., Goldstein, I. M., Snyderman, R., ed), 751-774, Raven Press. Ltd., New York, 1988) Similarly, in periodontitis or inflammation associated with the tooth-supporting periodontal ligament, the recruitment of neutrophils and monocytes from the circulation and the subsequent activation and release of ROS and proteolytic enzymes can eventually result in a significant and irreversible loss of underlying bone matrix at these sites. (Fredriksson, M., et al.., J Clin Periodontol 25(5), 394-8, 1998; Fredriksson, M. I., et al.., J Periodontal 70(11), 1355-1360, 1999) Despite the known participation of ROS in the inflammatory-mediated loss of underlying ECM, the mechanism(s) by which this occurs is not completely understood. This is particularly true for collagen, which is the major ECM protein in cartilage and bone.

Neutrophils and monocytes contain two enzymes that are responsible for producing ROS. The first is NADPH-oxidase, which catalyzes the formation of O₂ by the transfer of electrons from NADPH to oxygen via cytochrome b₅₅₈. (Babior, B. M., et al.., J Clin Invest 52(3), 741-4, 1973; Segal, A. W., et al.., Lancet 2 (8087), 446-9, 1978; Rossi, F. Biochim Biophys Acta 853(1), 65-89, 1986) Superoxide is rapidly dismutated to H₂O₂ either spontaneously or via the enzyme superoxide dismutase. (McCord, J. M., and Fridovich, I. J Biol Chem 243(21), 5753-60, 1968) Neither O₂ nor H₂O₂ exhibit significant reactivity with biologic compounds. (Gartner, A., Weser, U. Top. Curr. Chem. 132, 1-61 1986) The second enzyme is myeloperoxidase (MPO), which catalyzes the formation of HOCl from H₂O₂ and Cl⁻(Harrison, J. E., Schultz, J. J Biol Chem 251(5), 1371-4, 1976) MPO has been localized to the primary granules of resting neutrophils and the extracellular space and phagolysosomes of phagocytically-stimulated neutrophils. (Bainton, D. F., Farquhar, M. G. J Cell Biol 39(2), 299-317, 1968; Briggs, R. T., et al.., J Cell Biol 67(3), 566-86, 1975) MPO is also released from cytoplasmic granules of monocytes and some macrophages. (Hurst, J. K., Barrette, W. C., Jr. Crit Rev Biochem Mol Biol 24(4), 271-328, 1989) The cationic nature of MPO allows it to adhere to cell and matrix surfaces and localize to sites of inflammation. (Selvaraj, R. J., et al.., J Infect Dis 137(4), 481-5, 1978) It is at these sites that MPO produces HOCl, a highly reactive oxidant that readily reacts with primary amines to generate long-lived N-chloramines. (Weiss, S. J., et al.., J Clin Invest 70(3), 598-607, 1982; al., Science 222(4624): 625-628, 1983; Thomas, E. L., et al.., Methods Enzymol 132, 569-85, 1986) Although N-chloramines exhibit a lower oxidizing potential than HOCl, their much longer effective lifetime (˜18 hr) would enable them to cause damage at more distant sites than HOCl. (Thomas, E. L., et al.., Methods Enzymol 132, 569-85, 1986) Under acidic conditions, similar to the environment found in phagolysosomes, MPO generates Cl₂. (Hazen, S. L., et al.., J Clin Invest 98(6), 1283-9, 1996) MPO is also one of the pathways by which neutrophils generate O₂(¹Δg) (Steinbeck, M. J., et al . . . J Biol Chem 267(19), 13425-33, 1992).

Determining the ability of HOCl to contribute to the pathogenesis of inflammatory processes associated with rheumatoid arthritis and periodontitis is highly dependent on determining the relevant target(s) at these sites. The most likely protein target for neutrophil oxidants, including N-chloramines, is the ECM. ECM components found in or associated with articular cartilage or bone include hyaluronate, proteoglycans, fibronectin, several tissue specific and nonspecific protein components, and collagen, the major component of these tissues. It has been reported that neutrophil-generated ROS mediate the degradation of hyaluronate (Greenwald, R. A., Moak, S. A. Inflammation 10(1), 15-30, 1986) modify proteoglycan structure and/or synthesis, and alter the structure of fibronectin. (Greenwald, R. A., Moak, S. A. Inflammation 10(1), 15-30, 1986; Vissers, M. C., Winterbourn, C. C. Arch Biochem Biophys 285(1), 53-9, 1991; Vissers, M. C., Winterbourn, C. C. Arch Biochem Biophys 285(2), 357-64, 1991) Vissers et al . . . (1986) (Vissers, M. C., Winterbourn, C. C. Biochim Biophys Acta 889(3), 277-86, 1986) reported an increase in proteolytic degradation of glomerular basement membrane collagen by elastase in response to myeloperoxidase/H₂O₂/Cl⁻. Davies et al . . . (1993) (Davies, J. M., et al.., Free Radic Biol Med 15(6), 637-43, 1993) reported that >1.0 mM HOCl was required to cause extensive fragmentation of collagen type II isolated from bovine articular cartilage or collagen type I isolated from bovine tendon. Davies et al . . . (Davies, J. M., et al.., Free Radic Biol Med 15(6), 637-43, 1993) also reported that N-chloramines did not cause direct fragmentation, but greatly increased the degradation of collagen by collagenase and elastase. In general, there are very few studies available as to the susceptibility of collagen to oxidation by HOCl.

An important determinant for stability of the ECM is the degree of cross-linking. One important type of cross-link is pyridinoline (Pyd), which was first described by Fujimoto et al . . . (1978) (Fujimoto, D., et al.., Biochem Biophys Res Commun 84(1), 52-7, 1978) and later confirmed to be a non-reducible intermolecular cross-link of mature fibrillar collagen type I of bone. (Eyre, D. R. The Chemistry and Biology of Mineralized Connective Tissues (Veis, A., ed), 51-55, Elsevier North Holland, Inc., 1981). These cross-links were also found to be especially abundant in mature fibrillar collagen type II of articular cartilage (Eyre, D. R. Science 207, 1315-1322, 1980) where they covalently link collagen type II to other type II helical regions, collagen type IX to the surface of type II, and bind collagen type IX to other molecules of collagen type IX. Their function is to stabilize the collagen fibrillar superstructure, or arrays, and make them more resistant to collagenolysis or proteolytic degradation.(Vater, C., et al.., Biochem. J. 181, 639-645, 1979).

The present invention relates to the susceptibility of collagen cross-links and lysine/hydroxylysine to reaction with HOCl, Cl₂, and/or N-chloramine. Pyd cross-links were chosen as potential oxidation targets because of their importance in maintaining the collagen superstructure and because their chemical structure implies they are targets of oxidative modification by HOCl/Cl₂. These cross-links react with HOCl, Cl₂, and/or N-chloramines, thereby implying that Pyd is a site for ROS modification of collagen type I and II in bone and cartilage, respectively. The present invention relates to a method of detecting chlorinated products of collagen oxidation in bodily fluids, including but not limited to synovial fluid, serum and urine. The methods disclosed herein are also useful for monitoring of the efficacy of therapeutic agents in treating a diseased condition. Further, the invention disclosed herein includes a kit for use in detecting these oxidation/chlorination products for disease diagnosis, and use of this kit in monitoring the course of therapeutic agents in treating a diseased condition, for example rheumatoid arthritis, osteoarthritis ,etc.

### SUMMARY OF THE INVENTION

It is an object of the present invention to diagnose an inflammatory disease or condition associated with articular cartilage or bone surfaces in a mammal. A patient sample is obtained from the mammal and an amount of a chlorinated compound/peptide(s) is detected in the sample. In one embodiment the detecting step further comprises measuring the amount of the chlorinated compound/peptide(s) in the patient sample. In another embodiment the chlorinated compound/peptide(s) are at least one of the group of chlorinated forms of pyridinoline cross-links, lysine/hydroxylysine residues, aromatic amino acids, ketoimines and pyrole cross-links. In a further embodiment the method is an immunospecific binding of an antibody to the chlorinated compound/peptide(s) in the patient sample. In another embodiment the method is to detect the chlorinated compound/peptide(s) in the patient sample by U.V. absorbance or fluorescence In yet another embodiment the method is to detect the chlorinated compound/peptide(s) in the patient sample by mass spectrometry.

It is a further object of the present invention to monitor the course of therapeutic agent(s) in treating a diseased condition associated with articular cartilage or bone surfaces in a mammal. A patient sample is obtained from the mammal and an amount of a chlorinated compound/peptide(s) is detected and measured in the sample. In another embodiment the chlorinated compound/peptide(s) are at least one of the group of chlorinated forms of pyridinoline cross-links, lysine/hydroxylysine residues, aromatic amino acids, ketoimines and pyrole cross-links. In a further embodiment the method is an immunospecific binding of an antibody to the chlorinated compound/peptide(s) in the patient sample. In another embodiment the method is to detect the chlorinated compound/peptide(s) in the patient sample by U.V. absorbance or fluorescence In yet another embodiment the method is to detect the chlorinated compound/peptide(s) in the patient sample by mass spectrometry.

Another objective of the present invention is to provide a test kit for detecting and/or measuring a chlorinated compound/peptide(s) in a mammal. The kit contains, in one or more containers, a detectably labeled antibody immunospecific for the chlorinated compound/peptide(s).

### DESCRIPTION OF THE DRAWINGS

FIG. 1. Chemical structures of (A) pyridinoline, a cross-link involved in maintaining the interaction and structural integrity of collagen types I, II, III, IX, and XI, (B) pyridoxamine dihydrochloride, and (C) vitamin B₆, two chemical substitutes for pyridinoline. (B), UV absorbance peaks of 15 μM pyridoxamine or vitamin B₆ in 0.5 M sodium phosphate buffer, pH 7.2, are 217-219, 251-252, and 321-325 nm. (C), at pH 5.5, vitamin B₆ has an additional absorbance peak at 292 nm.

FIG. 2. The UV absorbance spectra of 15 μM pyridoxamnine in 0.5 M sodium phosphate buffer ±0.1 M NaCl, pH 7.2, reacted for 15 min at RT with 0 μM (-●-). 12.5 μM (- [symbol] -), 25 μM (-◯-) or 50 μM HOCl (-♦-) NOTE: A NUMBER OF SYMBOLS HAVE BEEN LOST IN FIGURE LEGENDS 2,4,5!!! in the presence of 0.1 M NaCl (A) and without NaCl present (B). The UV absorbance of pyridoxamine reacted for 15 min at RT with H₂O₂ or a10:1 mix of H₂O₂ and HOCl; (-Δ-), 500 μM H₂O₂; (-●-), 0 μM H₂O₂/HOCl; (- [symbol] -), 125 and12.5 μM H₂O₂/HOCl; (-◯-); 250 and 25 μM H₂O₂/HOCl, or (-♦-), 500 and 50 μM H₂O₂/HOCl in the presence of 0.1 M NaCl (C) or without NaCl present (D). Graphs are a representative set of data from experiments that were repeated three times.

FIG. 3. Fluorescence of 15 μM pyridoxamine in 0.5 M sodium phosphate buffer ±0.1 M NaCl, reacted for 15 min at RT with( [symbol] ) CT, ( [symbol] ) HOCl, ( [symbol] ) H₂O₂(1×10¹) or ( [symbol] ) H₂O₂ (1×10¹)/HOCl, at pH 7.2 in the presence of 0.1 M NaCl (A) or without NaCl present (B). Pyridoxamine reacted at pH 5.5 in the presence of 0.1 M NaCl (C) or vitamin B₆ in the presence of 0.1 M NaCl at pH 5.5 (D). After reaction, 3 ml of each sample are excited at 325 nm, and the emission intensity at 400 nm is determined. The fluorescence data are presented as the mean ± S.E. of a set of experiments repeated three times. Statistical significance of differences between control and treatment values is determined by a pairwise comparison of correlated groups using Student's t test, and statistical significance is defined as **p<0.01 or *p <0.05.

FIG. 4. The UV absorbance spectra of 15 μM pyridoxamine in 0.5M sodium phosphate buffer 0.1 M NaCl, pH 5.5, reacted for 15 min at RT with 0 μM (-●-), 12.5 μM (-), 25 μM (-◯-), or 50 μM HOCl (-♦-)in the presence or absence of 0.1 m NaCl (A) or 15 μM pyridox-amine reacted with 500 μM H₂O₂ (-Δ-), 0 μM H₂O₂/HOCl (-●-), 125 μM, 12.5 μM H₂O₂/HOCl (- [symbol] -), 250 μM, 25 μM H₂O₂/HOCl (-◯-), or 500 μM, 50 μM H₂O₂/HOCl (-♦-) in the presence or absence of 0.1M NaCl (B). The UV absorbance spectra of 15 μM vitamin B₆ in 0.5 M sodium phosphate buffer ±0.1 M NaCl, pH 5.5, reacted for 15 min at RT with 0 μM (-●-), 12.5 μM (- [symbol] -), 25 μM (-◯-), or 50 μM HOCl (-♦-) in the presence of 0.1 M NaCl (C) or without NaCl present (D). Graphs present a representative set of data from experiments that are repeated three times.

FIG. 5. The UV absorbance peaks for 0 μM (-●-), 25 μM (-◯-), 50 μM (- [symbol] -), 75 μM (-♦-), and 100 μM (-Δ-) chloramine-T in 0.5 M sodium phosphate buffer ±0.1 M NaCl at pH 7.2 (A) or pH 5.5(C). One major peak at 219-224 nm characterize the absorbance for chloramine-T. The UV absorbance for 15 μM pyridoxamine reacted with increasing concentrations of chloramine-T ±0.1 M NaCl for 15 min RT at pH 7.2 (B) or pH 5.5(C). The UV absorbance scans for 15 μM vitamin B₆ reacted with increasing concentrations of chloramine-T in 0.5 M sodium phosphate buffer pH 5.5±0.1 M NaCl for 15 min at RT (D), and then incubated overnight (16-18 h) at 37° C.

FIG. 6. Gas chromatography and electron ionization-mass spectrometry (GC-MS) of vitamin B₆ (A) and vitamin B₆ reacted with 50 μM HOCl for 15 min at pH 5.5 (B). Two peaks at 12.73 and 13.42 min characterize the GC-MS for vitamin B₆ (A), and four chlorinated products are identified at 11.03, 12.29, 13.17, and 14.19 min for vitamin B₆ (B) after reaction with HOCl and are identified as 4-chloro-2-hydroxymethyl-2,4-hexadiene-3-carboxaldehyde, 5-chloromethyl-3-hydroxy-4-hydroxymethyl-1,3,5-hexatriene, N-chloro-3-chloromethyl-4-hydroxy-2-hydroxy-methyl-1-imino-2,4-pentadiene, and 3-chloro-4,5-dihydroxymethyl-2-methylpyridine (3-chloropyridinium), respectively. The product at 13.57 min is not chlorinated, and no further characterization is carried out.

FIG. 7. 50 μg of human collagen type I or type II are incubated at pH 5.0 or pH 7.2 for 1 h at 37° C. in 0.5M sodium phosphate buffer (control) or buffer containing H₂O₂ and/or HOCl as indicated. After incubation, 0.1 μg of each sample is analyzed for carbonyl content. Only samples reacted with HOCl show increased carbonyl formation. The immuno-chemiluminescence (Oxy-blot kit) data is presented for a typical experiment performed three times. The bar graph shows the relative mean density ± S.E. for all three experiments.

FIG. 8. 50-μg samples of collagen type I (A) and collagen type II (B) are reacted at pH 5.0 and pH 7.2 or collagen types I or II are reacted at pH 5.0 (C) or pH 7.2 (D) for 1 h at 37° C. in 0.5 M sodium phosphate buffer alone or buffer containing H₂O₂ and/or HOCl as indicated. After ROS exposure, 4 μg of human collagen type I, type II, or [¹⁴C]-bovine serum albumin (¹⁴C-BSA) are subjected to SDS-PAGE analysis and stained with silver. E, ¹⁴C-BSA is reacted at pH 7.2 in buffer alone or buffer containing HOCl at 12.5 (lane 2), 25 (lane 3), or 50 μM (lane 4). Exposing collagens type I, type II, or [¹⁴C]-BSA to 12.5-50 μM HOCl results in a concentration-dependent decrease in the intensity of the electrophoretic band staining by silver (arrows) without causing a loss of protein as indicated by the single and equally intense (F) autoradiograph band for [¹⁴C]-BSA in lanes 1-4. Lanes marked M contain molecular mass markers (kDa). The presented data represent typical results of an experiment performed three times.

FIG. 9. Tryptic digest of the Cl-vasointestinal peptide (VIP) standard (25 μg) fractionated on a reverse-phase C₁₈ column using a 5% to 70% acetonitrile in 0.1% trifluoroacetic acid gradient over a period of 40 minutes and analyzed by Mass spectrometry for the presence of Cl⁻. (A) HPLC reverse phase separation profile of a tryptic digest of Cl-VIP peptide monitored at 214 nm and (B) positive ion-mass spectrum of the 20.68 min HPLC fraction (5-6 μg) of the N-peptide of Cl-VIP. The mass spectrum of the 20.68 min peak showed a strong (M+H) pseudomolecular ion at m/z 1203.5, with an isotope pattern consistent with the presence of Cl

### DESCRIPTION OF THE INVENTION

Experimental Procedures

Pepsin-solubilized collagen types I and II isolated from human bone and articular cartilage, respectively, are kindly provided by Dr. David Eyre (University of Washington, Seattle, Wash.). Pyridoxamine dihydrochloride, pyridoxine hydrochloride, 30% hydrogen peroxide, Chloramine-T, 1,4-dimethylnaphthalene, methylene blue, and all other salts and buffer components are purchased from Sigma-Aldrich Chemical Company (St. Louis, Mo.). Glacial acetic acid and dichloromethane are purchased from Fisher Scientific (Fairlawn, N.J.). Potassium iodide is purchased from Acros Organics (Fairlawn, N.J.). Disodium anthracene-9,10-dipropionic acid is purchased from Molecular Probes (Eugene, Oreg.). Sodium hypochlorite (NaOCl) (4-5% available chlorine) is purchased from Sigma-Aldrich Chemical Company (St. Louis, Mo.), Acros Organics (Fairlawn, N.J.), Fisher Scientific (Fairlawn, N.J.), Alfa (Ward Hill, Mass.), and Cole-Palmer (Vernon Hills, Ill.); see comments about NaOCl below.

Preparation of Pyridoxamine and Vitamin B₆.

10 mM stock solutions of pyridoxamine dihydrochloride (pyridoxamine) and pyridoxine hydrochloride (Vitamin B₆) are made fresh weekly and stored at 4° C. with protection from light. Stock solutions are made in one of the following buffers: 0.5 M glacial acetic acid buffer pH 3, or 0.5 M sodium phosphate buffer pH 5.5±0.2, pH 7.2±0.2, or pH 8.0±0.2. Where appropriate, 0.1 M NaCl is added to the buffers as a source of chloride ions.

Control and Reactive Oxygen Species Reaction with Pyridoxamine and Vitamin B₆.

On the day of use, stock solutions of pyridoxamine and Vitamin B₆ are diluted in appropriate buffer to 20 μM. 7.5 ml of the 20 μM solutions of pyridoxamine or Vitamin B₆ are then added to control and treated sample tubes so the final concentration of pyridoxamine or Vitamin B₆ in all samples is 15 μM in a total volume of 10 ml. Samples are brought to a total volume of 10 ml by the addition of 2.5 ml of appropriate buffer to control samples or 1.25 ml to hydrogen peroxide (H₂O₂) and sodium hypochlorite (NaOCl) samples. No buffer is added to samples treated with the combination of H₂O₂ and NaOCl (H₂O₂/NaOCl). H₂O₂ and NaOCl treatment solutions are prepared by diluting the stock solution of H₂O₂ to 1, 2, or 4 mM, and diluting the stock solution of NaOCl to 0.1, 0.2, or 0.4 mM in appropriate buffer immediately before use. 1.25 ml of a diluted solution of H₂O₂ or NaOCl, or 1.25 ml each of diluted solutions of H₂O₂ and NaOCl are added to appropriate tubes. The final concentrations of H₂O₂ are 125, 250, or 500 μM, and the final concentrations of NaOCl are 12.5, 25, or 50 μM. H₂O₂ is always added first to either the H₂O₂ or H₂O₂/NaOCl samples and NaOCl is added immediately after, where appropriate. Samples are mixed after each addition. At pH 12, the NaOCl stock solution exists predominantly as the conjugate base, hypochlorite (⁻OCl).

H₂O₂ (30% or 12.92M) and NaOCl (0.65M) stock solutions as supplied by the manufacturer are stored tightly sealed at 4° C. with protection from light. Despite these precautions, both stock solutions decomposed over a period of 3-4 months after being opened. In general, decomposition of NaOCl could be identified by a yellowing of the solution. Yellowing is always accompanied by a distinctively greater reactivity of the NaOCl solutions, which is attributed to the breakdown of NaOCl to Cl⁻ and eventually reactive chlorate (ClO₃⁻).(Sidgwick, N. V. The chemical elements and their compounds, II, Clavendon Press, Oxford, 1950; Sneed, M. C., et al.., Comprehensive inorganic chemistry, Van Nostred Co. Inc., NY, 1954; Downs, A. J. and Adams, C. J., Comprehensive inorganic chemistry,1400-1408, Pergamon Press, Oxford, 1973). Therefore, caution should be exercised when using NaOCl, since even new solutions made by different companies show signs of decomposition.

U.V. Absorbance and Fluorescence.

After the addition of buffer, H₂O₂, NaOCl, or H₂O₂/NaOCl, control and treated samples are incubated for 15 min at RT (approximately 25° C.) in a Precision Scientific Low Temperature Incubator 815 (Chicago, Ill.). At the end of each incubation, the U.V. absorbance of a 50 μl aliquot of each sample is scanned from 400 to 200 nm in a Beckman DU-640 Spectrophotometer (Fullerton, Calif.), and the fluorescence intensity of a 3 ml aliquot of each sample is read in a Perkin Elmer Fluorescence Spectrophotometer (Norwalk, Conn.). Optimal fluorescence excitation and wavelengths are determined by referring to U.V. absorbance peaks and by pre-scanning samples for maximal excitation and peaks. The known excitation and peak wavelengths for both pyridoxamine and Vitamin B₆ are 324 nm excitation and 400 nm. A 200 μl aliquot of each control and treated sample is also stored at 4° C. after incubation for later N-chloramine analysis.

N-chloramine Assay.

The presence of N-chloramines is determined by the method of Witko et al.. (1992). (Witko, V., et al., J Clin Lab Anal 6(1), 47-53, 1992) This method is based on the colorometric measurement of triiode ions formed by the oxidation of potassium iodide (KI) in solution. Chloramine-T (N-chloro-p-toluene-sulfonamide sodium salt), a commercially available source of N-chloramine, is used to calibrate the assay. A 100 mM stock solution of chloramine-T is made fresh weekly in distilled H₂O and stored at 4° C. with protection from light. The 100 mM chloramine-T solution is then diluted in appropriate buffer to final concentrations of 25, 50, 75, or 100 μM immediately before use. The RT incubation is extended from 2-min (Witko, V., et al., J Clin Lab Anal 6(1), 47-53, 1992) to 5 min without a significant difference in results. The direct oxidation of KI by H₂O₂, NaOCl, or H₂O₂/NaOCl is also determined, and these values are subtracted as background from the correspondingly treated samples. The resulting difference represented the amount of N-chloramine present in each sample.

Chloramine-T Reactivity.

On the day of use, 10 mM stock solutions of pyridoxamine or Vitamin B₆ are diluted to a concentration of 1 mM, and a 100 mM stock solution of chloramine-T is diluted to final concentrations of 25, 50, 75, or 100 μM in appropriate buffer. Control and chloramine-T treated samples contained 15 μl of the diluted 1 mM pyridoxamine or Vitamin B₆ solution plus 1 ml of appropriate buffer (control) or 1 ml of 25, 50, 75, or 100 μM chloramine-T solution. The final concentration of pyridoxamine or Vitamin B₆ for all control and chloramine-T treated samples is 15 μM. Following preparation, samples are incubated for 15 min at RT. At the end of each incubation, the U.V. absorbance of a each sample is scanned from 400 nm to 200 nm to look for any changes in the absorbance of pyridoxamine or Vitamin B₆ due to a reaction with chloramine-T and to identify the absorbance peaks for N-chloramines and pyridoxamine-chloramine or Vitamin B₆-chloramine reaction products. In addition, the N-chloramine assay of Witko et al.. (Witko, V., et al., J Clin Lab Anal 6(1), 47-53, 1992) is performed to determine if a reaction of chloramine-T with pyridoxamine or Vitamin B₆ occurs, as indicated by a decrease in the amount of chloramine-T available to oxidize KI.

1,4-Dimethyl-1,4-Naphthalene Endoperoxide (DNE) Synthesis and Release of O₂(¹Δg).

DNE, a pure chemical source of O₂(¹Δg) that thermally releases O₂(¹Δg) at 37° C., is synthesized by the method of Wasserman and Larsen (1972). (Wasserman. H. H., Larsen, D. L., J. Chem. Soc. Chem. Commun. 5, 253-255, 1972; Steinbeck, M. J., et al., J Histochem Cytochem 41(11), 1659-67, 1993) A duplicate set of coverslips are coated with either dichloromethane or a solution of DNE in dichloromethane (3.6 mg/100 μl) by surface evaporation at 4° C. 200 μl of either a 15 μM pyridoxamine or Vitamin B₆ solution in appropriate buffer is added to a duplicate set of dichloromethane-(control) and DNE-coated coverslips. One set of control and DNE-coated coverslips is incubated overnight at 4° C. and another at 37° C. The release of O₂(¹Δg) from DNE is confirmed after overnight incubation at 37° C. by following the decrease in absorbance at 400 nm of anthracene-9,10-diproprionic acid (AAP) (1×10⁻⁴M) in 0.5M sodium phosphate buffer, pH 7.2, according to the method of Deby-Dupont et al. (Deby-Dupont, G., et al., Biochim Biophys Acta 1379(1), 61-8, 1998).

Gas Chromatography-Mass Spectrometry.

Gas chromatography-mass spectrometry (GC-MS) using electron ionization-mass spectrometry (EI-MS) are performed by M-Scan, Inc., West Chester, Pa. In brief, reacted samples containing Vitamin B₆ alone or Vitamin B₆ in a 1:1 ratio with NaOCl are lyophilized, dissolved in 40 μl in dimethyl formamide and silylated derivatives prepared by the addition of 100 μl of TMS (N,O-bis (trimethylsilyltrifluoro-acetamide with trimethylchlorosilane: Supelco, Pa. 16823) followed by heating to 35° C. for 5 min. Derivatized products are concentrated to ˜50 μl under anhydrous N₂, and analyzed on capillary column (Perkin Elmer PE-5MS, 30 m×0.25 mm×25 μm) by GC-MS (Perkin Elmer Auto System XL Gas chromatograph with Perkin Elmer Turbomass Quadrupole mass spectrometer) in the positive electron ionization mode. El-MS is used to identify the structure of individual compounds in each GC peak. The source and interface temperatures are both 200° C. The injector temperature is maintained at 280° C., and the initial GC oven temperature is 70° C. for 2 min followed by an increase to 140° C./min to 300° C.

Preparation of Pepsin-Solubilized Collagen.

The lyophilized collagen samples are dissolved in 0.5M acetic acid at a concentration of 1.2 mg/ml overnight at 4° C. with gentle stirring and protection from light. Samples are then dialyzed against 0.02M dibasic sodium phosphate buffer, pH 9.0 for 48 hr at 4° C. using a dialysis cassette made by Pierce Slide-A-lyzer (Pierce Chemical Company, Rockford, Ill.) according to manufacturers instructions. The samples are split into two equal volumes and dialyzed for an additional 48 hr at 4° C. with protection from light against 0.5M sodium phosphate buffer to bring the pH to approximately 5.0±0.3 or 7.2±0.3. After dialysis, samples are removed from cassettes and stored at 4° C. until use. The collagen suspensions are turbid and contained fibrils and/or a variety of polymorphic forms of collagen in equilibrium with monomers. These collagen preparations represent a mixture of fibrils consisting of cross-linked trimers, dimers, and α-subunits of collagen (α,. β, and γ. bands, respectively) visulaized by PAGE. (Brodsky, B., and Eikenberry, E. F., Methods Enzymol 82, 127-74, 1982; Piez, K. A., Extracellular Matrix Biochemistry (Piez, K. A., and Reddi, A. H., ed), 1-39, Elsevier Science Publishing Co., Inc., New York, 1984). At pH 7.2, collagen type II preparations also contained a small amount of aggregate/particulate material, also in equilibrium with monomers. The collagen preparations do not require sonication and are easily suspended and reproducibly loaded onto nitrocellulose or into wells for SDS-PAGE fractionation. (SanAntonio, J. D., et al., J Cell Biol 125(5), 1179-1188, 1994).

Reactive Oxygen Species Treatment of Collagen.

On the day of use, 50 μg of collagen type I or II are added to 1.5 ml eppendorf tubes. Control samples receive appropriate buffer only and ROS-treated samples received appropriate buffer containing H₂O₂, HOCl, or the combination of H₂O₂ and HOCl (H₂O₂/HOCl). Final concentrations of H₂O₂ are 125, 250, or 500 μM; final concentrations of HOCl are 12.5, 25, or 50 μM in a total volume of 200 μl. Samples are mixed after each addition and then incubated for 1 hr at 37° C.

Oxyblot for the Detection of Carbonyl (Aldehyde and Ketones) Formation.

Carbonyl groups are formed as a consequence of protein oxidation and in the reaction of HOCl with pyridinium compounds. The 2,4-dinitrophenylhydrazine assay for carbonyls (Levine, R. L., et al., Methods Enzymol 186, 464-78, 1990) is performed according to kit instructions in the Oxyblot oxidized protein detection kit (Oncor, Gaithersburg, Md.) without SDS-PAGE separation. In brief, aliquots of each collagen sample are reacted with 2,4-dinitrophenylhydrazine to derivatize carbonyl groups to the product 2,4-dinitrophenylhydrazone. After derivatization, aliquots of each collagen sample are diluted in SDS-PAGE sample buffer, spotted onto dry nitrocellulose, and the derivatized product is detected by chemiluminescence using a horseradish peroxidase-conjugated antibody that specifically recognizes 2,4-dinitrophenylhydrazone. The spot intensities are quantified by scanning densitometry (Arcus II flatbed scanner; AFGA) using NIH Image version 1.57 software (Wayne Rasband, National Institutes of Health, Bethesda, Md.).

Acetone Precipitation.

Cold precipitation of collagen samples is performed according to the Pierce BCA Applications Note #13 (Pierce Chemical Company, Rockford, Ill.). 200 μl of −20° C. acetone are added to 50 μl of each sample, vortexed, and placed at −20° C. for 30 min. The samples are then centrifuged at 12,000×g for 10 min in a microcentrifuge at 4° C., supernatants are removed, and the remaining acetone is evaporated by leaving samples uncovered for 30 min at RT.

Fluorescamine and O-Phthalaldehyde (OPA) Primary Amine and Imino Acid Measurements.

Fluorescamine reacts directly with primary amines or imino acids to yield highly fluorescent derivatives that emit fluorescence at 475 nm when excited at 390 nm (Udenfriend, S., et al., Science 178(63), 871-2, 1972) and is used according to the method of Bohlen et al.. (1973). (Bohlen, P., et al., Arch. Biochem. & Biophy. 155(1), 213-20, 1973) O-phthalaldehyde also reacts with primary amines and imino acids and is used according to manufacturer instructions (Pierce Chemical Company, Rockford, Ill.). Both assays are performed on aliquots of each collagen sample, acetone precipitates of each sample, or sample supernatants of acetone precipitates after ROS and/or protease treatments. Triplicates of each sample (200 μl) are placed in a 96-well cytoplate (CFCPN9610, Millipore Corporation; Bedford, Mass.) and the fluorescence is read at an excitation wavelength of 340±20 nm and an emission wavelength of 400±20 nm (Cytofluor 2350, Perseptive Biosystems, Inc.; Cambridge, Mass.).

SDS-PAGE Electrophoresis.

4 μg aliquots of acetone precipitated collagen or non-precipitated collagen samples are resuspended in SDS-PAGE sample buffer and subjected to electrophoresis using a 5% stacking gel and a 10% separating gel prepared according to a modified Laemmli procedure previously described in detail (Steinbeck, M. J. et al., J Biol Chem 266(25), 16336-42, 1991) or using precast linear gradient gels (4-15% acrylamide) purchased from Bio-Rad Laboratories (Hercules, Calif.). The gels are stained with silver using the Bio-Rad silver stain kit (Bio-Rad Laboratories, Hercules, Calif.). The collagen gels are quantified by scanning densitometry (Arcus II flatbed scanner; AFGA). Band intensities are analyzed using NIH Image version 1.57 software (Wayne Rasband, National Institutes of Health, Bethesda, Md.). To determine the reactivity of silver with protein after reaction with HOCl, ¹⁴C-bovine serum albumin is reacted with increasing concentrations of HOCl under the conditions described above for collagen. After reaction, the ¹⁴C-bovine serum albumin is subjected to gel electrophoresis, stained with silver, dried and exposed to x-ray film. Silver staining intensity of each band is then compared with audoradiogram band intensity of the same gel.

Data Analysis.

Data are expressed as the mean ± SEM. To evaluate the treatment effects, the data are grouped by experiment and time point for statistical analysis. Statistical significance of differences between the vehicle only (control) and treatment values for an individual experiment and time point is determined by a pairwise comparison of correlated groups using Student's t-test from the GB-STAT statistics software version 5.4.1.

High Performance Liquid Chromatography (LC)-Mass Spectrometry (MS)

LC-MS using positive ion-MS are performed by M-Scan, Inc., West Chester, Pa. In brief, a model chlorinated peptide, chlorinated vasointestinal peptide (Cl-VIP) (Sigma, St. Louis, Mo.) is used as an internal standard to determine the appropriate parameters for detecting chlorine-containing peptides in patient samples (such as, but not limited to, synovial fluid, serum and urine). The signal output from the standard peptide is further enhanced by digesting Cl-VIP with trypsin prior to use. The tryptic digest of the Cl-VIP standard is then fractionated, using standard HPLC methodology, on a reverse-phase C₁₈ column using a 5% acetonitrile to 70% acetonitrile in 0.1% trifluoroacetic acid gradient over a period of 40 minutes. Those HPLC fractions containing protein (as determined by measuring UV absorbance at 214 nm) are further analyzed by positive ion-MS. The mass spectrum peaks are then scanned for the presence of chlorinated peptides using the Micromass software search (MassLynx).

Generation of Antibodies

In order to generate antibodies specific for the chlorinated products of the present invention (including, but not limited to,4-chloro-2-hydroxymethyl-2,4-hexadiene-3-carboxaldehyde, 5-chloromethyl-3-hydroxy-4-hydroxymethyl-1,3,5-hexatriene, N-chloro-3-chloromethyl-4-hydroxy-2-hydroxymethyl- 1-imino-2,4-pentadiene, and 3-chloro-4,5-dihydroxymethyl-2-methylpyridine (3-chloro-pyridinium) (infra) and lysine residues), collagen peptides containing the chlorinated cross-links and lysine or hydroxylysine residues are isolated. In general, the procedures include, but are not limited to, the following. Collagen types I and II are reacted with HOCl at pH7.2 or 5.5 followed by treatment with collagenase, elastase, and/or cathepsin K (supra). A sequence of chromatographic steps are used to isolate the individual peptides containing oxidized and chlorinated pyridinoline, pyrole. ketoimine, lysine, hydroxylysine and aromatic amino acids (tyrosine/phenylalanine). These include, but are not limited to, adsorption on selective cartridges of a hydrophobic interaction support and an ion-exchange support and molecular sieve or ion-exchange and reverse-phase HPLC column chromatography steps. Intact pyridinoline cross-links are detected by monitoring the fluorescence of these groups at 400 nm in the individual column fractions and confirmed by mass spectrometry. Detection of chlorinated peptides is done by analyzing the HPLC fractions by mass spectrometry. These procedures are well known to those skilled in the art. See, for example, Malle, E., et al.. Arteriosclerosis, Thrombosis & Vascular Biology 15(7): 982-989, 1995.

The purified chlorinated compounds/peptides are used in the generation of both monoclonal and polyclonal antibodies specifically binding the chlorinated products and their equivalents disclosed herein. Antibodies are prepared by methods known in the art. For example see Campbell, A. M. Laboratory Techniques in Biochemistry and Molecular Biology, Vol. 13 (1986), Elsevier. It is possible to produce antibodies to the above chlorinated compounds/peptides or their equivalents as isolated. However, because the molecular weights of these peptide fragments are generally less than 5,000, it is preferred that the hapten be conjugated to a carrier molecule. Suitable carrier molecules include, but are not limited to, bovine serum albumin, ovalbumin, thyroglobulin, and keyhole limpet hemocyanin (KLH).

A number of binding agents may be suitably employed. These include, but are not limited to, carbodiimides, glutaraldehyde, mixed anhydrides, as well as both homobifunctional and heterobifunctional reagents (see for example the Pierce 1986-87 catalog, Pierce Chemical Co., Rockford, Ill.).

Methods for binding the hapten to the carrier molecule are known in the art. See for example, Chard, T., Laboratory Techniques in Biochemistry and Molecular Biology, Vol. 6 (1987) Partz Elsevier, N.Y.

Either monoclonal or polyclonal antibodies to the hapten-carrier molecule immunogen are produced. However, it is preferred that monoclonal antibodies (MAb) be prepared. For preparation of monoclonal antibodies directed toward the chlorinated products any technique which provides for the production of antibody molecules by continuous cell lines in culture may be used. For example, the hybridoma technique originally developed by Kohler and Milstein (Nature 256:495-497, 1975), as well as the trioma technique, the human B-cell hybridoma technique (Kozbor et al . . . Immunology Today 4:72, 1983), and the EBV-hybridoma technique to produce human monoclonal antibodies (Cole et al.., in Monoclonal Antibodies and Cancer Therapy. Alan R. Liss, Inc., pp. 77-96, 1985).

In an additional embodiment of the invention, monoclonal antibodies can be produced in germ-free animals utilizing recent technology (PCT/US90/02545). Immunization protocols for the mouse usually include an adjuvant. Various adjuvants used to increase the immunological response, include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

Examples of suitable protocols are well known to those of skill in the art. Spleen cells from the immunized mouse are harvested and homogenized and thereafter fused with cancer cells in the presence of polyethylene glycol to produce a fused cell hybrid which produces monoclonal antibodies specific to chlorinated products. Suitable cancer cells include myeloma, hepatoma, carcinoma, and sarcoma cells. Detailed descriptions of this procedure, including screening protocols, protocols for growing selected hybrid cells and harvesting monoclonal antibodies produced by the selected hybrid cells are provided in Galfre, G. and Milstein, C., Meth. Enzymol., 73:1 (1981).

Detection and Quantitation of Chlorinated Products in Patient Samples

Immunological binding partners, especially monoclonal antibodies, produced by the above procedures, or equivalent procedures, are employed in various immunometric assays to detect and quantitate the concentration of the chlorinated products in patient samples. The chlorinated product(s) can be that present in one of many different species, including but not limited to, mammalian, bovine, ovine, porcine, equine, rodent and human. Such samples include, but are not limited to, synovial fluid, serum, and urine. These immunometric assays comprise a monoclonal antibody or antibody fragment coupled to a detectable label. As used herein, the term detectable label refers to any label which provides, directly or indirectly, a detectable signal. Examples of suitable detectable labels include, but are not limited to: enzymes, coenzymes, enzyme inhibitors, chromophores, fluorophores, and chemiluminescent materials. Examples of standard immunometric methods suitable for quantitating the chlorinated products include, but are not limited to, enzyme immunoassays such as, for example, enzyme-linked immunosorbant assay, immunoenzymatic assays, enzyme-multiplies immunoassay technique, substrate-labeled fluorescent immunoassay, and fluorescence polarization immunoassay. The present invention provides a method for detecting a chlorinated product(s) in a patient sample by contacting the patient sample with an antibody directed to the chlorinated product(s) under conditions such that immunospecific binding occurs, and detecting and/or measuring the amount of any immunospecific binding by the antibody.

Other methods of detecting the chlorinated compound/peptide(s) of the present invention include, but are not limited to, mass spectrometry, flurometry, U.V. absorption and potassium iodide assays (see supra).

An additional aspect of the present invention relates to diagnostic kits for the detection and/or measurement of chlorinated products. Kits for use in diagnostic and therapeutic monitoring are provided, that comprise in one or more containers an antibody to each of the chlorinated products, and, optionally, a labeled binding partner to the antibody. Alternatively, the antibody can be labeled with a detectable marker, e.g., a chemiluminescent, enzymatic, or fluorescent (supra).

Results

U.V. Absorbance and Fluorescence Emission of Pyridoxamine and Vitamin B₆.

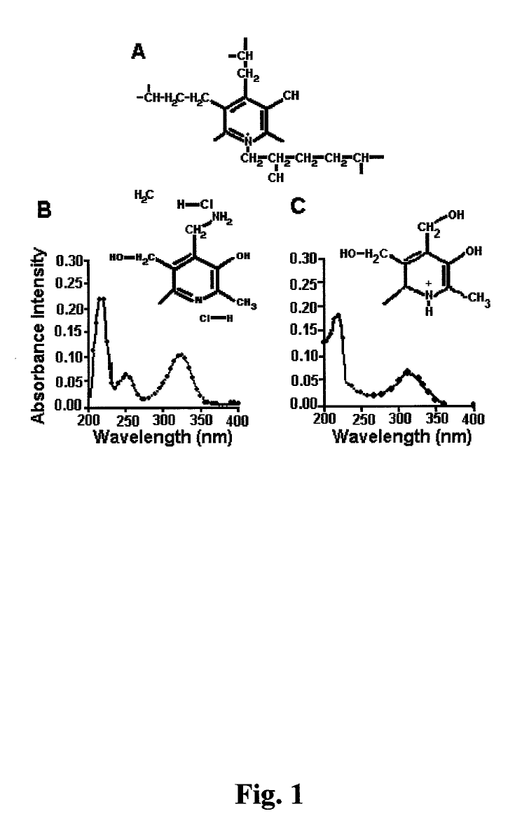

Pyridoxamine dihydrochloride (FIG. 1B) and Vitamin B₆ (FIG. 1C) share the pyridinium ring structure and spectral properties of the pyridinoline (Pyd) trifunctional cross-links (FIG. 1A) of collagens, including collagen types I, II, III, IX, and XI. The spectral properties of pyridoxamine, Vitamin B₆, and Pyd include their characteristic U.V. absorbance and excitation maximum at 325 nm, pH 7.2 (Fig. 1B). The absorbance characteristics of pyridoxamine and Vitamin B₆ include three peaks at 217-219, 251-252, and 321-325 nm. At pH 5.5, a hydrogen ion binds to the nitrogen group (Brealey, G. J., Kasha, M. J. Am. Chem. Soc. 77, 4462-4468, 1955) of the pyridinium ring of Vitamin B₆, resulting in an additional peak at 292 nm (FIG. 1C).

Oxidative Modification of Pyridoxamine and Vitamin B₆ After Exposure to the ROS, H₂O₂, HOCl, or O₂(¹Δg).

To evaluate the oxidation of pyridoxamine or Vitamin B₆ by H₂O₂, HOCl, or O₂(¹Δg), a 15 μM solution of pyridoxamine or Vitamin B₆ dissolved in 0.5 M sodium phosphate buffer, pH 7.2±0.2, is exposed for 15 min at RT to 125, 250, or 500 μM of H₂O₂, 12.5, 25, or 50 μM of HOCl, or a combination of H₂O₂/HOCl in a ratio of 10:1. The above concentrations of HOCl and H₂O₂ are within the predicted range generated by activated neutrophils or monocytes at sites of inflammation. (Klebanoff, S. J. Inflammation: Basic Principles and Clincal Correlates (Gallin, J. I., Goldstein, I. M.. Snyderman, R., ed), 391-444, Raven Press, Ltd., New York, 1988) Concentrations of H₂O₂ and HOCl in this range are possible within specialized microenvironments, such as phagocytic vacuoles or the neutrophil- or macrophage-attachment sites, because the aqueous volumes in these microenvironments are thought to be nanoliters or less, resulting in μM to mM concentrations of reactive oxygen species (ROS). (Berger, R. R., Karnovsky, M. L. Fed Proc 25(3), 840-5, 1966) A ratio of 10:1 is used because the amounts of HOCl generated by activated neutrophils is 5-20× less than relative amounts of H₂O₂ generated by the same cells stimulated under the same condition.( Klebanoff, S. J. Inflammation: Basic Principles and Clincal Correlates (Gallin, J. I.. Goldstein, I. M., Snyderman, R., ed), 391-444, Raven Press, Ltd., New York, 1988)

O₂(¹Δg) is generated when both HOCl and H₂O₂ are added together, equation 1, or when HOCl is added in buffer containing Cl⁻ pH 7.2, equation 2:

1) H₂O₂+HOCl→O₂(¹Δg)+HOH+Cl⁻+H⁺ (Khan, A. U., Kasha, M. J. Chem. Phys. 39, 2105-2106, 1963)

2) 2HOCl→O₂(¹Δg)+2Cl⁻+2H⁺ (Khan, A. U., Kasha, M. Proc Natl Acad Sci U S A 91(26), 12362-4, 1994)

The amount of O₂(¹Δg) generated in the reaction of H₂O₂ with HOCl is pH dependent, with the greatest amount of O₂(¹Δg) being produced at alkaline pH, intermediate amounts at neutral pH, and essentially non-measurable amounts at acidic pH due to assay limitations and interference by chlorine (Cl₂). No O₂(¹Δg) is produced by HOCl in the absence of Cl⁻ or at pH<4.2 or ≧8.0.(Khan, A. U., and Kasha, M. Proc Natl Acad Sci U S A 91(26), 12362-4, 1994)

At pH 7.2, HOCl would exist in almost equal concentrations with ⁻OCl (pKa=7.5) (Held, A. M., et al., J. Am. Chem. Soc. 100, 5732-5740, 1978) and acidification of HOCl (pH below 6.0, peaking at pH 5.25) in the presence of Cl⁻ results in the evolution of chlorine (Cl₂), equation 3:

3) HOCl+Cl⁻+H⁺→HOH+Cl₂ (Sidgwick, N. V. The chemical elements and their compounds, II, Clavendon Press, Oxford, 1950; Khan, A. U., and Kasha, M. J. Chem. Phys. 39, 2105-2106, 1963; Held, A. M., et al., J. Am. Chem. Soc. 100, 5732-5740, 1978)

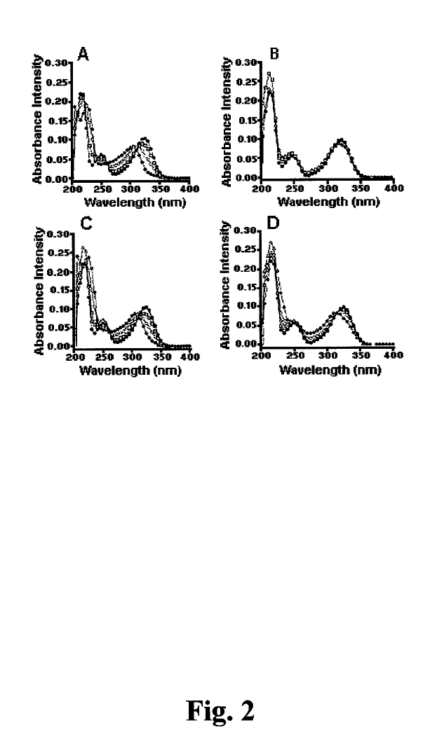

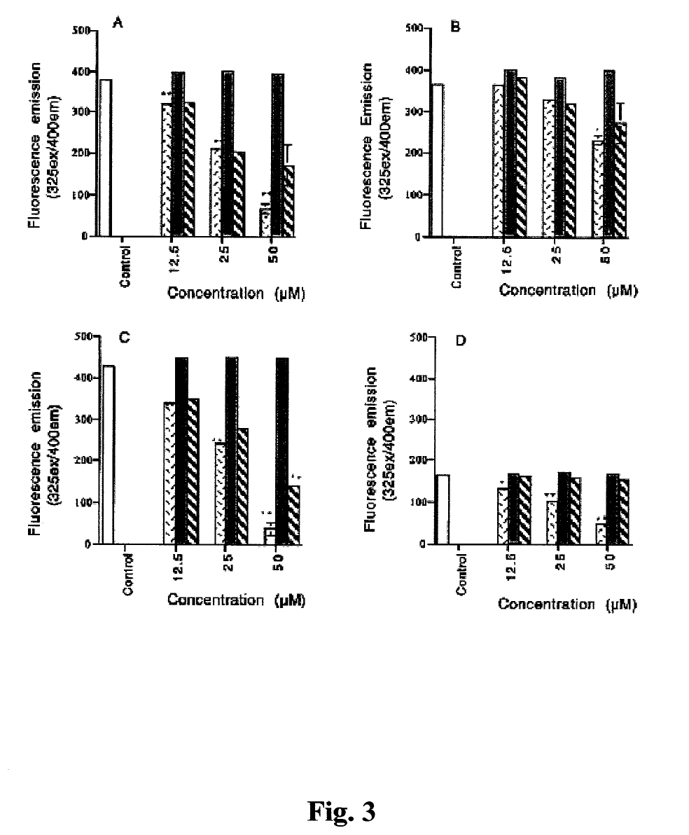

The U.V. absorbance data for a typical experiment using 0.5M sodium phosphate buffer containing 0.1M NaCl, pH 7.2, is presented in FIG. 2. The U.V. absorbance scans of pyridoxamine treated with HOCl show an immediate concentration-dependent shift in maximum absorbance at 217 nm and 325 nm (FIG. 2A). Accompanying the shift in U.V. absorbance in response to increasing concentrations of HOCl is the appearance of two new absorbance peaks at 220-225 nm to and 307-320 nm. In parallel, pyridoxamine excitation at 325 nm and fluorescence at 400 nm is dramatically decreased in response to increasing concentrations of HOCl. The fluorescence data for HOCl-treated samples are presented in FIG. 3A as the mean ± SEM (n=3, **p<0.01, *p<0.05). No other changes in fluorescence excitation or emission are observed. These findings follow the predicted increase in reactivity rate of HOCl in the presence of Cl⁻.( Sidgwick, N. V. The chemicaI elements and their compounds, II, Clavendon Press, Oxford, 1950; Sneed, M. C., et al., Comprehensive inorganic chemistry, Van Nostred Co. Inc., NY, 1954; Bailar, J. C., Jr., et al., Comprehensive inorganic chemistry, Pergamon Press, Oxford, 1973: Latimer, W. M. The oxidation states of the elements and their potentials in aqueous solutions, Prentice-Hall Inc., NY, 1938).

The combination treatment of H₂O₂/HOCl also shifts the absorbance (FIG. 2C) and decreases the fluorescence (FIG. 3A) of pyridoxamine. The absorbance and fluorescence changes in response to H₂O₂/HOCl are similar to the changes observed when pyridoxamine is treated with the corresponding concentration of HOCl alone. When 0.1M NaCl is omitted from the buffer system, only minor effects on the absorbance and fluorescence are observed in response to either HOCl (FIG. 2B & 3B) or H₂O₂/HOCl (FIG. 2D & 3B). H₂O₂ alone had no effect on the U. V. absorbance or fluorescence of pyridoxamine in either buffer system (FIG. 2C-D & 3A-B), although the absorbance of H₂O₂ around 219 nm contributes to the slight increase in absorbance at this wavelength in samples treated with this ROS.

As shown in FIG. 1, Vitamin B₆ has essentially the same chemical structure as pyridoxamine, except for a —CH₂OH group in the para position of the pyridinium ring instead of the —CH₂NH₂ group. This difference is important because a reaction of HOCl with the primary amine, —CH₂NH₂, of pyridoxamine is favored at pH 7.2 (Thomas, E. L., et al., J Clin Invest 72(2), 441-54, 1983) over a reaction with the nitrogen of the pyridinium ring. The reaction of HOCl with the primary amine of pyridoxamine will produce an N-chloramine/—CH₂NHCl group/ N-chloro-pyridoxamine. In contrast to HOCl, the reactivity of O₂(¹Δg) with the nitrogen of the pyridinium ring has been reported. (Straight, R. C., and Spikes, J. D. Singlet O₂ (Frimer, A. A., ed) Vol. IV, pp. 91-143, I-IV vols., CRC Press, Inc., Boca Raton, Fla., 1985) Despite the potential of O₂(¹Δg), and possibly HOCl, to react with the ring nitrogen, there is essentially no reaction of H₂O₂ or H₂O₂/HOCl and little reaction of HOCl with Vitamin B₆ in the presence or absence of NaCl, although the degree to which HOCl reacted with Vitamin B₆ varied.

Oxidation of Pyridoxamine and Vitamin B₆ at pH 5.5±0.2.

The pH of the phagolysosomes of neutrophils during the first 15 min following the ingestion of opsonized particles is 7.4-7.8 (Cech, P., and Lehrer, R. I. Blood 63(1), 88-95, 1984; Segal, A. W., et al., Nature 290(5805), 406-9, 1981) but after 15 min, the pH within the phagolysosomes, and presumably the underlying extracellular attachment site, decreases to pH 5.5-6.0. pH 5.5 is also the optimal pH for the generation of Cl₂ and O₂(¹Δg) by HOCl in solutions containing 0.1M NaCl. (Khan, A. U., Kasha, M. Proc Natl Acad Sci U S A 91(26), 12362-4, 1994; Held, A. M., et al., J. Am. Chem. Soc. 100, 5732-5740, 1978)

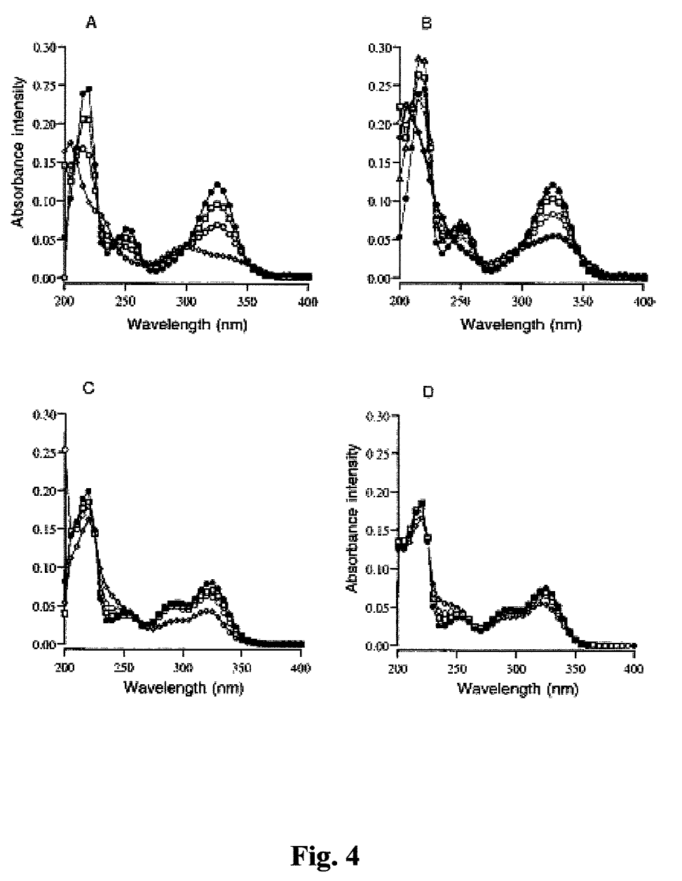

To evaluate the reactivity of HOCl, H₂O₂, or H₂O₂/HOCl with pyridoxamine and Vitamin B₆ at pH 5.5±0.2, ROS are added to 15 μM pyridoxamine in 0.5M sodium phosphate buffer ±0.1M NaCl. Pyridoxamine or Vitamin B₆ control and treated samples are then incubated at RT for 15 min. The U.V. absorbance data for a typical pyridoxamine experiment is presented in FIG. 4. At pH 5.5 the absorbance of pyridoxamine at 217 and 325 nm, and to a lesser extent at 252 nm, decreases after treatment (FIG. 4A-B), and the formation of a new absorbance peak at 228-229 nm is detected, which is within the range of known N-chloramine absorbance peaks. (Hazen, S. L., et al., J Clin Invest 98(6), 1283-9, 1996)

At pH 5.5, HOCl also reacts with Vitamin B_{6,} which results in a decrease in the absorbance at 292 and 325 nm (FIG. 4C). In the absence of NaCl, Vitamin B₆ samples treated with HOCl show only a slight decrease in absorbance in response to 50 μM HOCl (FIG. 4D), implying that either Cl₂ or O₂(¹Δg) are involved in this reaction.

The fluorescence data for all samples are presented in FIG. 3C-D as the mean ± SEM (n=3, **p<0.01, *p<0.05). The fluorescence of pyridoxamine (FIG. 3C) and Vitamin B₆ (FIG. 3D) at 400 nm is decreased in response to either HOCl or H₂O₂/HOCl treatments. The decrease in absorbance (FIG. 4) and fluorescence (FIG. 3) of both pyridoxamine and Vitamin B₆ implies that the ring structure is disrupted by this reaction.

N-Chloramine Formation in HOCl-Treated Samples.

To determine whether the reaction of HOCl with pyridoxamine or Vitamin B₆ leads to the formation of N-chloramines, 15 μM pyridoxamine or Vitamin B₆ in 0.5M sodium phosphate ±0.1M NaCl pH 7.2 are reacted with increasing concentrations of HOCl. The oxidation of potassium iodide (KI) to triiodide by HOCl or by N-chloramines formed in the reaction of HOCl with pyridoxamine is determined after incubation for 15 min at RT.

In the absence of NaCl, HOCl in solution at pH 7.2 is slow to decompose and the direct oxidation of KI by HOCl remains high, even after 24 hr incubation at 37° C. (Table I). Due to the high background, the formation of N-chloramines by HOCl in the absence of NaCl can not be determined, although an immediate reaction of HOCl with pyridoxamine is observed as a decrease in KI oxidation in these samples.

In the presence of NaCl, there is an immediate decrease in background oxidation of KI by unreacted HOCl and the production of N-chloramines is detected after 15 min in pyridoxamine samples reacted with 50 μM HOCl (Table I). As would be expected from the limited effect of HOCl on the absorbance and fluorescence of Vitamin B_{6,} there is no formation of N-chloramines in the reaction of HOCl with Vitamin B₆ at pH 7.2.

Despite the more rapid decomposition of HOCl or the rapid reaction of HOCl with NaCl at pH 5.5, the formation of N-chloramines can not be determined at this pH. The increased oxidation of KI under these conditions may be due to the production of O₂(¹Δg).

Table I. N-chloramine formation determined by the oxidation of potassium iodide after the reaction of pyridoxamine with HOCl (0.5M sodium phosphate, pH 7.2). The data are presented as the mean ± SEM of a set of experiments repeated three times. Statistical significance of differences between control and treatment values was determined by a pairwise comparison of correlated groups using Student's t-test and statistical significance is defined as **p<0.01 or *p<0.05.

| | HOCl | | | |
|---|---|---|---|---|
| ADDITIONS: | 0 μM | 12.5 μM | 25 μM | 50 μM |
| No NaCl | 0 | 13.4 ± 1.2 | 24.3 ± 1.0 | 48.0 ± 3.2 |
| 0.1 M NaCl | 0 | 9.4 ± 0.5 | 18.2 ± 2.0 | 3.1 ± 0.4** |
| 15 μM | 0.6 ± 1.1 | 0.5 ± 0.5 | 0.4 ± 1.1 | 15.1 ± 2.5* |
| Pyridoxa- | | | | |
| mine + 0.1 M | | | | |
| NaCl | | | | |

Reaction of Chloramine-T with pyridoxamine and Vitamin B₆.

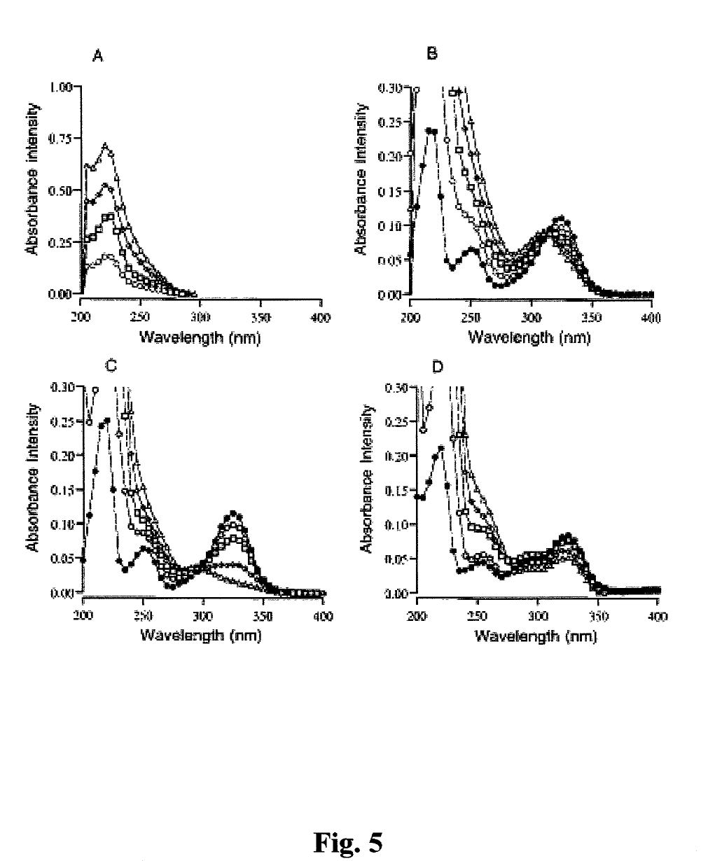

To further evaluate the formation of N-chloro-pyridoxamine in the reaction of HOCl with pyridoxamine, the absorbance of chloramine-T in 0.5M sodium phosphate buffer, pH 7.2 and 5.5, is determined (FIG. 5A). The absorbance peaks for chloramine-T are 219-224 nm, and correspond with the initial absorbance peaks of HOCl-treated pyridoxamine samples (FIG. 2), indicating the reaction of HOCl with pyridoxamine generates N-chloro-pyridoxamine. The absorbance of unreacted chloramine-T remains constant at both pHs.

To evaluate the reaction of N-chloramines with pyridoxamine or Vitamin B₆, increasing concentrations of chloramine-T are added to 15 μM pyridoxamine or Vitamin B₆ in 0.5M sodium phosphate buffer, pH 7.2 or 5.5, and the samples are incubated for 15 min RT, 2 hr at 37° C., or overnight at 37° C. Chloramine-T reacts with pyridoxamine at pH 7.2 as indicated by the concentration-dependent shifts in the major absorbance peak of pyridoxamine from 325 nm to 312-318 nm (FIG. 5B-C). There is essentially no reaction of chloramine-T with Vitamin B₆.

At pH 7.2, the reaction of chloramine-T with pyridoxamine is verified using the N-chloramine assay (supra). There is a decrease over time in the amount of chloramine-T available to oxidize KI after reaction with pyridoxamine, but not Vitamin B₆ at pH 7.2. The reaction of chloramine-T with pyridoxamine is immediate. and continues to increase over time.

At pH 5.5, chloramine-T reacts with both pyridoxamine (FIG. 5C) and Vitamin B₆ (FIG. 5D), although the Vitamin B₆ reaction is not immediate. The reactivity of chloramine-T with Vitamin B₆ implies that the nitrogen in the pyridinium ring is reactive at this pH, although to a lesser degree compared with the primary amine group of pyridoxamine.

Thermal release of O₂(¹Δg) from DNE.

DNE, a pure chemical source of O₂(¹Δg), will provide a reaction system that does not rely on the presence of other ROS's that may interfere or react with the target of interest, thereby allowing a more definitive evaluation of the reaction of O₂(¹Δg) with pyridoxamine or Vitamin B₆. 200 μl of 15 μM pyridoxamine or Vitamin B₆ in 0.5M sodium phosphate containing 0.1M NaCl, pH 7.2 or 5.5, is incubated on a DNE-coated coverslip. Duplicate samples are incubated for 24 hr at 4° C. or 37° C., and the absorbance readings are compared. Control coverslips are coated with the dichloromethane solvent. There are no changes in the absorbance of either pyridoxamine or Vitamin B₆ in response to O₂(¹Δg). The release of O₂(¹Δg) is verified using anthracene-9,10-dipropionic acid (AAP). (Deby-Dupont, G., et al., Biochim Biophys Acta 1379(1), 61-8, 1998). After 24 hr at 4° C., the absorbance of AAP at 400 nm is 0.715±0.003 and AAP incubated on a DNE-coated coverslip had an absorbance of 0.702±0.005. For duplicate samples incubated at 37° C., the absorbance of AAP after 24 hrs on control coverslips is 0.718±0.003 and the absorbance of AAP incubated on DNE-coated coverslips is 0.588±0.008. The decrease in absorbance of AAP on DNE-coated coverslips after incubation at 37° C. confirms that O₂(¹Δg) is released and reacts with AAP in the aqueous buffer systems used in the present invention. Taken together these findings eliminate O₂(¹Δg) in the reaction of HOCl with either pyridoxamine or Vitamin B₆ at pH 5.5, leaving Cl₂ and HOCl as the remaining reactants.

Gas chromatography and mass spectrometry.

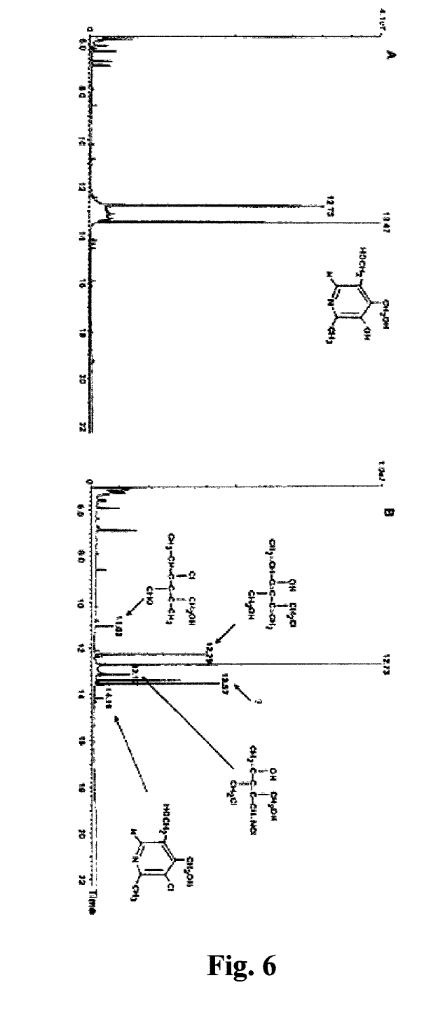

Samples of Vitamin B₆ and Vitamin B₆ reacted with 50 μM HOCl for 15 min are analyzed by GC-MS (supra). FIG. 6 presents the results of these analyses. Electron ionization-mass spectrometry of the Vitamin B₆-GC peak shows two peaks at 12.73 and 13.42 (FIG. 6A). After reaction with 50 μM HOCl at pH 5.5 (FIG. 6B), four chlorinated products are identified at 11.03, 12.29, 13.17, and 14.19 min as 4-chloro-2-hydroxymethyl-2,4-hexadiene-3-carboxaldehyde, 5-chloromethyl-3-hydroxy-4-hydroxymethyl-1,3,5-hexatriene, N-chloro-3 -chloromethyl-4-hydroxy-2-hydroxymethyl-1-imino-2,4-pentadiene, and 3-chloro-4,5-dihydroxymethyl-2-methylpyridine (3-chloro-pyridinium), respectively. The product at 13.57 min is not chlorinated and no further characterization is carried out.

Carbonyl (aldehyde and ketone) formation after exposure of collagen to H₂O₂, HOCl, or H₂O₂/HOCl.

To evaluate the oxidation of human collagen types I (bone) and II (articular cartilage) by H₂O₂, HOCl, or H₂O₂/HOCl, 50 μg of collagen in 0.5 M sodium phosphate buffer containing 0.1M NaCl, pH 7.2 or pH 5.0, are exposed for 1 hr at 37° C. to 125, 250, or 500 μM H₂O₂, 12.5, 25, or 50 μM HOCl, or a combination of H₂O₂/HOCl in a ratio of 10:1. The above concentrations of HOCl and H₂O₂ are within the predicted range generated by activated neutrophils or monocytes at sites of inflammation. (Klebanoff, S. J. Inflammation: Basic Principles and Clincal Correlates (Gallin, J. I., Goldstein, I. M., Snyderman, R., ed), 391-444, Raven Press, Ltd., New York, 1988) A ratio of 10:1 is used because the amounts of HOCl generated by activated neutrophils is 5-20X less than relative amounts of H₂O₂ generated by the same cells stimulated under the same conditions. (Klebanoff, S. J. Inflammation: Basic Principles and Clincal Correlates (Gallin, J. I., Goldstein, I. M., Snyderman, R., ed), 391-444, Raven Press, Ltd., New York, 1988) The pHs are chosen based on the neutral pH of the neutrophil phagolysosomes during the first 15 min following the ingestion of opsonized particles (Cech, P., and Lehrer, R. I. Blood 63(1), 88-95, 1984; Segal, A. W., et al., Nature 290(5805), 406-9, 1981) and the acidic pH after 15 min.( Lukacs, G. L., et al., J Biol Chem 265(34), 21099-107, 1990)

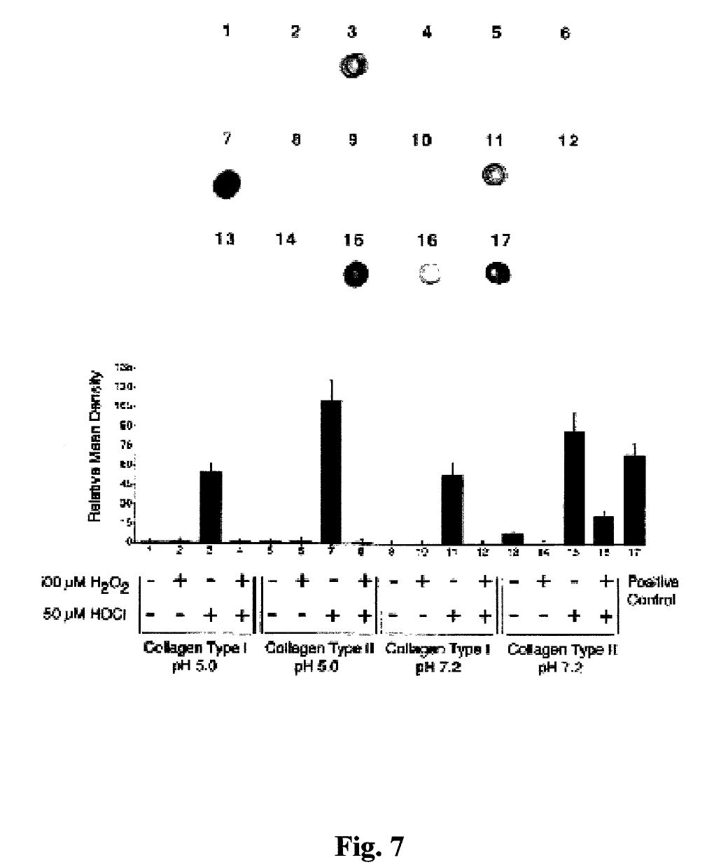

To determine the oxidation of primary amines or pyridinoline cross-links by H₂O₂ or HOCl, immuno-chemiluminescence analysis (Oxyblot kit) is used to assess the presence of carbonyl groups within the protein of interest. A concentration-dependent increase in the number of reactive carbonyl groups in both collagen types is found after exposure to HOCl, with the amount varying depending on collagen type. The results for collagen reacted with 500 μM H₂O₂, 50 μM HOCl, or both are presented in FIG. 7. Lesser amounts of carbonyls are generated in the reaction of 25 μM and 12.5 μM HOCl with both collagen types. In general, collagen type I is less reactive at both pH 5.0 and pH 7.2 as compared to collagen type II, as indicated by relative mean density (FIG. 7).

No change in carbonyl content is observed relative to untreated collagen control samples after treatment of either collagen type with H₂O₂ or a combination of H₂O₂/HOCl (FIG. 7). Adding H₂O₂ and HOCl together decreases the effect of HOCl. suggesting that H₂O₂ is reacting with HOCl and decreasing the availability of HOCl to react with collagen.

Pridinoline cross-link and aromatic amino acid fluorescence of collagen before and after exosure to HOCl.

To assess oxidation of the pyridinoline cross-links and aromatic amino acids of collagen types I and II by HOCl, the fluorescence emission of HOCl-treated collagen is measured and compared to untreated control samples subjected to the same conditions. The fluorescence of collagen type I samples is below measurable levels, and no further fluorescence studies are done on this collagen type. Exposure of 0.125 mg/ml of collagen type II to HOCl at pH 5.0 and 7.2 for 15 min at 37° C. results in a concentration-dependent decrease in fluorescence emission at 400 nm (excitation 325 nm) (Table II). No other changes in fluorescence emission or excitation are observed.

Table II. Exposure of human articular collagen type II to HOCl resulted in a decrease in fluorescence emission at 400 nm (excitation 325 nm) after a 15 min incubation at 37° C. Data are presented as a percentage decrease in fluorescence emission relative to unreacted collagen incubated under the same conditions. Values represent the mean ± SEM (n=2).

| | HOCl | | |
|---|---|---|---|
| | 1 μM | 3 μM | 4 μM |
| 0.125 mg/ml collagen | 8.5 ± 5.0% | 9.4 ± 4.5% | 10.0 ± 4.5% |
| pH 5.5 | | | |
| 0.125 mg/ml collagen | 10.0 ± 5.7% | 18.5 ± 7.0% | 21.0 ± 6.0% |
| pH 7.2 | | | |
| *Pyd: HOCl | 1:1.2 | 1:3.5 | 1:4.6 |

*Ratio of Pyd: HOCl, assuming the amount of 2 mol Pyd/mol collagen remains unchanged by pepsin-solubilization of the collagen type II.

SDS-PAGE results of collagen types I or II reacted with H₂O₂, HOCl, or a combination of H₂O₂/HOCl.

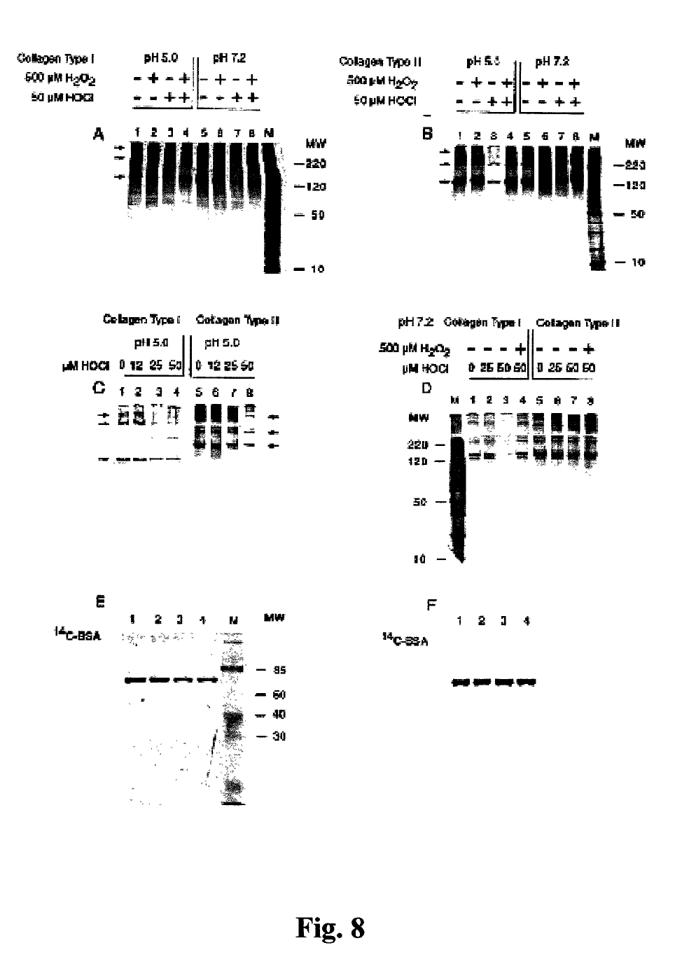

After exposure to ROS (supra), 4 μg aliquots of each collagen sample are subjected to SDS-PAGE analysis (4-15% gradient) and stained with silver. Results of a representative gel of an experiment performed three times are presented in FIG. 8. Exposing collagen types I (FIG. 8A, lanes 3 and 7) or II (FIG. 8B, lane 3) to 50 μM HOCl resulted in a 70-80% decrease in the intensity of collagen electrophoretic band staining by silver (arrows indicating form top to bottom, (α, β, and γ). Except at pH 7.2, the decrease in silver staining for collagen type II reacted with HOCl is much less (FIG. 8B, lane 7). Concentration-dependent decreases in silver staining are shown in FIGS. 8C and 8D for collagen reacted with HOCl at pH 5.0 and 7.2. No smearing (fragmentation) or low molecular weight bands are observed after any treatment. A decrease in reactivity of silver with the HOCl-reacted collagen monomers, is consistent with the formation of N-chloroamines (Davies, J. M., et al., Free Radic Biol Med 15(6), 637-43, 1993) in the reaction of HOCl with collagen, and ultimately the spontaneous deamination and decarboxylation of N-chloramines to form aldehydes. (Thomas, E. L., et al., Methods Enzymol 132, 569-85, 1986; Hazen, S. L., et al., Biochemistry 37, 6864-6873, 1998) Thus implying that the collagen monomers are still intact, but have been oxidatively modified and no longer react with silver. The oxidative modification of amine groups by HOCl will no longer make them available for reaction with silver.

The primary amines of lysine and hydroxylysine are reaction targets of HOCl/Cl₂. The reaction sites are the free nucleophilic groups (i.e. —NH₂/ε-amino group; pKa 10.54) of lysine residues or hydroxylysine residues, as determined by the decreased intensity of silver staining of collagen and ¹⁴C-BSA after reaction with HOCl/Cl₂. The decreased reactivity of the electrophilic silver (Ag²⁺) with nucleophilic groups of lysine and hydroxylysine is due to a prior reaction of the nucleophilic groups of lysine/hydroxylysine with HOCl/OCl- (pH 7.2) or Cl₂ (pH 5.5).

To determine if the decrease in silver reactivity with collagens after oxidation by HOCl, is due to oxidative modification of primary amine groups and not protein fragmentation or both, ¹⁴C-labeled bovine serum albumin (BSA) is reacted with increasing concentrations of HOCl (12.5-50 μM). After exposure to HOCl (infra), 4 μg aliquots of ¹⁴C-BSA are subjected to SDS-PAGE analysis (10% acrylamide) and stained with silver, then dried and exposed to autoradiograph film. Silver staining and autoradiogram results are presented in FIG. 8C and 8D, respectively. A concentration-dependent decrease in silver staining intensity is observed after treatment of ¹⁴C-BSA with 12.5-50 μM HOCl, without affecting the autoradiogram band intensity of these same samples. Thus providing evidence that the decrease in collagen staining after reaction with HOCl is due to oxidative modification rather than protein fragmentation or a combination of both processes.

Fluorescamine and o-phthalaldehyde primary amine and imino acid measurements.

To determine if the decrease in detectable electrophoretic band staining is the result of complete collagen fragmentation by HOCl, aliquots of each collagen sample acetone precipitates of each sample, and sample supernatants of acetone precipitates are analyzed by fluorescamine and o-phthalaldehyde assays. Both assays detect primary amines and imino acids, which will increase if collagen is fragmented. However, neither assay detects an increase in free amine or imino acid groups in any of the collagen samples.

High Performance Liquid Chromatography (LC) and mass spectrometry (MS)

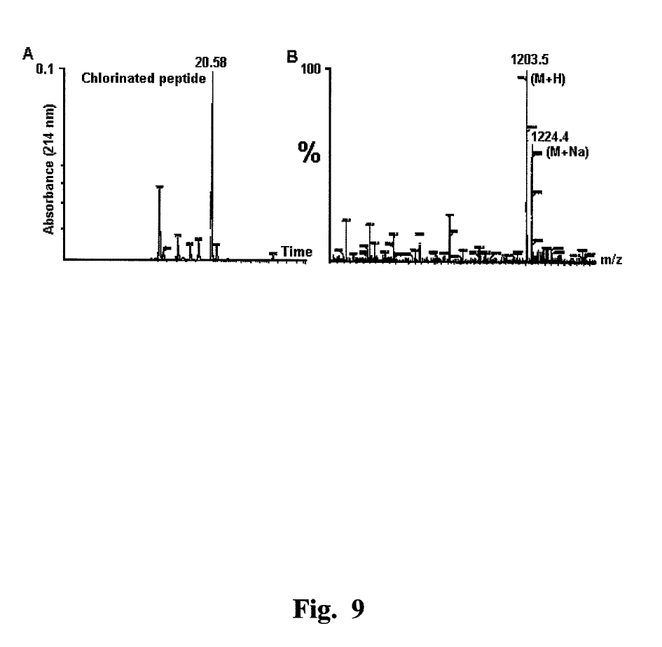

The tryptic digest of the Cl-VIP standard (surpa) is fractionated on a reverse-phase C₁₈ column (FIG. 9A). Those LC fractions containing protein are further analyzed by positive ion-MS. The mass spectrum peaks are then scanned for the presence of chlorinated peptides using the Micromass software search (MassLynx) (FIG. 9B). A number of digest products are detected by LC fractionation, including the N-terminal chlorinated peptide, HSDAVF(Cl)TDNY eluting at 20.68 mm. The mass spectrum of this peptide demonstrates a strong (M+H) pseudomolecular ion at m/z 1203.5, with an isotope pattern consistent with the presence of Cl.

Preparation of synovial fluid samples for analysis.

Synovial fluid is difficult to analyze because of its high viscosity and the presence of large amounts of serum albumin. To decrease viscosity and reduce the amount of albumin, several novel digestion methods are used in the present invention. Initial digestion is done using hyaluronidase, which digests the glycosaminoglycan in hyaluronic acid, the major component of synovial fluid. Hyaluronidase catalyzes the breakdown of the β(1-4) linkages of hyaluronic acid into its component parts, D-glucuronic acid and N-acetyl-D-glucosamine and thereby decreases the viscosity of the sample. The samples are also treated with trypsin and cyanogens bromide (CNBr) to degrade albumin, the second most abundant component of synovial fluid. Trypsin, which catalyzes the hydrolysis of peptide bonds for arginine and lysine residues, and CNBr, which specifically cleaves peptide bonds after methionine residues, are both used to ensure the degradation of albumin and the specific degradation of other proteins that might be present in the synovial fluid sample.

Aliquots of each sample are incubated overnight with 25 milliUnits of the hyaluronidase. To 25 μl of each sample, 10 μl of enzyme (2.5 U/μl in phosphate buffered saline) is added and the digests are incubated for 12 hr at 37° C. on a mixer. These aliquots are then further digested with trypsin. Tryptic digestion involves incubation of each sample with trypsin [1 mg/ml] (1:50 ratio of enzyme to sample) in 50 mM ammonium biocarbonate buffer, pH 8.3 for 24 hrs at 37° C. Finally, the aliquots undergoe CNBr treatment. This is performed by incubating the aliquots for 6 hrs at room temperature in the presence of CNBr. Under a fume hood, the aliquots are dissolved in a minimum amount of 70% formic acid and 2-3 crystals of CNBr are added to the dissolved aliquots. The samples are covered and incubated in a fume hood at room temperature for 6 hr. Distilled water is added to stop reaction and the sample is stored at −20° C. Thus, the present invention presents a novel method of digesting synovial fluid samples to allow for further testing to determine if Cl-peptides or other biomarkers are present.

Liquid Chromatography-Mass Spectroscopy (LC-MS) analysis of synovial fluid obtained from patients with varying degrees of joint injury

LC-MS are used to analyze the synovial fluid from patients with degenerative joint disease (DJD). The synovial fluids from three patients with early or advanced osteoarthritis (OA) were analyzed and compared with synovial fluid taken from a patient with an acute cruciate ligament (ACL) tear. An enzymatic digest of the synovial fluid samples from each patient is analyzed for the presence of chlorinated peptide(s). A number of protein digest products are detected by LC fractionation and the amount of protein in each fraction is determined by measuring the UV absorbance at 214 nm. Those LC fractions containing protein are further analyzed by positive ion-MS. The mass spectrum peaks are then scanned for the presence of chlorinated peptides using the Micromass software search (MassLynx). The patient samples are also analyzed for the presence of myeloperoxidase (MPO), the enzyme that is responsible for generating the chlorinated products (Bioxytech MPO-Enzyme Immunoassay, OxisResearch, Portland, Oreg.). Neither the control (001) nor the advanced OA patient samples (003 and 004) had detectable Cl-peptides (Table III). Only patient 002 diagnosed with early OA was positive for the presence of a Cl-peptide (Table III). The mass spectrum of this peptide demonstrated a strong (M+H) pseudomolecular ion at m/z 808.3, with an isotope pattern consistent with the presence of Cl. Patient 002 also had an elevated MPO level (Table III), which is consistent with the presence of a Cl-peptide. The elevated MPO activity in patient sample 004 is due to the amount of blood present in that sample, indicating that MPO alone is not an adequate biomarker for early DJD. The present invention thus provides a novel method of detecting the presence of Cl-peptides in body samples, such as, but not limited to, synovial fluid taken from patients with a degenerative joint disease, thereby serving as a biomarker for the early diagnosis of diseases associated with cartilage and bone surfaces.

TABLE III
The MPO and Cl-peptide results of four synovial fluid samples, and the
relevant patient information that may contribute to the onset or severity of OA.

| Synovial | Fluid | | | | | Cl-peptides | |
|---|---|---|---|---|---|---|---|
| Fluid | volume | | Race/ | | | /Molecular | MPO |
| Sample # | / | Diagnosis | sex | Height/Weight | Age | Mass (Da) | ng/ml |
| 001 | 10 ml | Torn ACL | W/m | 72″/185 lbs | 36 | Negative | 0 |
| 002 | 1 ml | Early OA | B/f | 62″/282 lbs | 31 | Positive/ | 52 |
| | | | | | | 808.3 Da | |
| 003 | 2 ml | Advanced | W/f | 76″/145 lbs | 63 | Negative | 10 |
| | | OA | | | | | |
| 004 | 3 ml | Advanced | W/f | 68″/200 lbs | 76 | Negative | 40 |
| | Bloody | OA | | | | | |
| | tap | | | | | | |

Discussion

ROS are produced and released into extracellular spaces and contribute to the development and progression of inflammatory diseases. However, little is known about direct ROS-induced molecular modifications of individual matrix proteins and the consequence of these modifications on the structure, physical properties, or function of the matrix. The present invention tests the hypothesis that HOCl participates in the inflammatory-mediated loss of connective tissue collagen by oxidizing the Pyd cross-links found in abundance in adult articular cartilage. HOCl. produced by the enzyme MPO, is the major highly reactive oxidant produced by activated neutrophils and to a lesser extent by monocytes and some macrophages. Based on its reactivity, HOCl has the potential to cause the bulk of the tissue damage at sites of acute inflammation. (Domigan, N. M., et al., J. Biol. Chem. 270, 16542-16548, 1995). Pyd cross-links are of particular importance because their function is to help maintain the structure of the collagen fibrils and make them more resistant to collagenolysis or proteolytic degradation. (Vater, C., et al., Biochem. J. 181, 639, 1979; Eyre, D. R., et al., Annu Rev Biochem 53, 717-48, 1984). The present invention reveals that HOCl/Cl₂, and to a lesser extent N-chloramines, chlorinate pyridinium compounds with structures similar to Pyd, and that HOCl reacts with both collagen types I and II, resulting in the oxidation of amine groups and Pyd cross-links.

HOCl rapidly reacts with and chlorinates pyridinium compounds, but the chlorination reaction and the N-chlorination sites are pH-dependent. At pH 7.2±0.2, HOCl/⁻OCl preferentially reacts with the para-CH₂NH₂ group of pyridoxamine to form N-chloro-pyridoxamine, which is detected using the KI oxidation assay for N-chloramine and by the appearance of the characteristic N-chloramine absorbance peak at 220-225 nm. In pyridoxamine samples treated with HOCl, a second absorbance peak at 307-320 nm is observed. This peak corresponds to a peak observed for the product that results from the reaction of pyridoxamine with chloramine-T, a commercial N-chloramine standard. Based on the presence of these two peaks, the data of the present invention imply that as soon as N-chloro-pyridoxamines are formed in the reaction of pyridoxamine and HOCl, they in turn react with other pyridoxamine molecules and initiate the formation of N-chloro-pyridoxamine dimers. The reaction of HOCl/⁻OCl with the para-CH₂NH₂ of pyridoxamine is in agreement with the report of Davies et al.. (1993) (Davies, J. M., et al., Free Radic Biol Med 15(6), 637-43, 1993) stating that the preferred reaction of HOCl/⁻OCl with collagen at neutral pH is with the primary amine groups.

At pH 5.5±0.2 and in the presence of Cl⁻, HOCl and N-chloramines react with both pyridoxamine and Vitamin B₆. The reactivity of HOCl at this pH with the pyridinium compounds is consistent with the reported increase in reactivity of HOCl with compounds that possess extensive π electrons (ring nitrogen). (Albrich, J. M., et al., Proc Natl Acad Sci U S A 78(1), 210-4, 1981) The significant loss in absorbance and fluorescence of either compound after reaction with HOCl at pH 5.5, implies that a percentage of the pyridinium ring structure is disrupted in these reactions. Disruption of the ring structure is confirmed by GC-MS analysis and is consistent with the formation of an aldehyde as a result of the spontaneous deamination and decarboxylation of an N-chloramine (in this case the ring nitrogen) to form an aldehyde.( Thomas, E. L., et al., J Clin Invest 72(2), 441-54, 1983; Hazen, S. L., et al., Biochemistry 37, 6864-6873, 1998)

The reactivity of HOCl with Vitamin B₆ at pH 5.5 in the presence of Cl⁻ is also consistent with the evolution of Cl₂. (Khan, A. U., and Kasha, M. Proc Natl Acad Sci U S A 91(26), 12362-4, 1994) GC-MS analysis also identifies 3-chloro-pyridinium as one of the products of this reaction, similar to a previous report of 3-chlorotyrosine formation in the reaction of Cl₂ with the aromatic amino acid, L-tyrosine. (Hazen, S. L., et al., J Clin Invest 98(6), 1283-9, 1996) The reactivity of Cl₂ is also of interest because it has been demonstrated that neutrophils generate Cl₂ via the MPO—H₂O₂—Cl⁻ system. (Albrich, J. M., et al., Proc Natl Acad Sci U S A 78(1), 210-4, 1981)

In contrast to HOCl, H₂O₂ alone is without effect under any condition, which is in keeping with its low reactivity with biological molecules. The third of the non-radical species tested, O₂(¹Δg), is a relatively long-lived (2 μsec) and highly reactive oxidant produced by HOCl at pH 5.5 and to a lesser extent at pH 7.2 in the presence of Cl⁻.( Khan, A. U., and Kasha, M. Proc Natl Acad Sci U S A 91(26), 12362-4, 1994) Because O₂(¹Δg) will be produced by both of these ROS systems, it seems likely that O₂(¹Δg) will react with the pyridinium ring nitrogen. (Straight, R. C., and Spikes, J. D. Singlet O₂(Frimer, A. A., ed) Vol. IV, pp. 91-143, I-IV vols., CRC Press, Inc., Boca Raton, Fla., 1985) However, O₂(¹Δg) does not significantly contribute to the derivatization of pyridoxamine and at pH 7.2 the production of O₂(¹Δg) by the reaction of H₂O₂ with HOCl/⁻OCl actually interferes with the reaction of HOCl and pyridoxamine. Similarly, at pH 5.5 HOCl should generate maximal amounts of O₂(¹Δg) in the presence of NaCl (Khan, A. U., and Kasha, M. Proc Natl Acad Sci U S A 91(26), 12362-4, 1994) however, even at this pH the present invention indicates that HOCl and not O₂(¹Δg) is the major reactant with either pyridoxamine or Vitamin B₆. Finally, the thermal release of O₂(¹Δg) by DNE, a pure chemical source of O₂(¹Δg), does not result in an absorbance or fluorescence change in the pyridoxamine or Vitamin B₆ spectra. It has been reported that O₂(¹Δg) can react with pyridinium compounds, resulting in cleavage of the nitrogen-carbon bond between the nitrogen-containing pyridinium ring and the terminal carbon of a substituted group at this site. (Straight, R. C., and Spikes, J. D. Singlet O₂ (Frimer, A. A., ed) Vol. IV, pp. 91-143, I-IV vols., CRC Press, Inc., Boca Raton, Fla. 1985) This reaction leaves the pyridinium ring structure intact and unchanged. Although a reaction of O₂(¹Δg) with an unsubstituted nitrogen group in the pyridinium rings of pyridoxamine and Vitamin B₆ is not detected, it is still possible that a reaction between O₂(¹Δg) and the Pyd cross-links can take place in vivo when the nitrogen is covalently linked to the triple helical region of a collagen molecule. This type of reaction will also result in the disruption of the intermolecular bond between two molecules of collagen.

In a previous study by Davies et al.. (1993) (Davies, J. M., et al., Free Radic Biol Med 15(6), 637-43, 1993) the oxidation of bovine collagen type I isolated from tendon and collagen type II isolated from articular cartilage was assessed as the amount of collagen fragmentation taking place in response to HOCl or N-chloramines. Only at super-physiological concentrations of 1-5 mM did HOCl cause extensive fragmentation (smearing) of collagen. In contrast, the addition of N-chloramines (5-50 μM) did not cause fragmentation, but instead, greatly increased the degradation of collagen by collagenase and elastase. The mechanism by which N-chloramines increased the proteolytic susceptibility of collagen was not specifically determined, although it was assumed that N-chloramines were reacting with amine groups and disrupting the secondary and tertiary structure of the collagen molecules. In many cases, oxidation of proteins appears to result in the partial unfolding of target proteins, exposing hydrophobic regions that are normally shielded and promoting the preferential degradation of these proteins by proteases. (Cervera, J., and Levine, R. L. FASEB J. 2, 2591-2595, 1988; Giulivi, C., et al., Arch. Biochem. Biophys. 311, 329-341, 1994; Pacifici, R. E., et al., J. Biol. Chem. 268, 15405-15411, 1993) Oxidation and disruption of these critical cross-links could result in the loss of functional interactions of collagen, destabilization of the structural integrity of collagen fibrils, and/or an increase the susceptibility of collagen to proteolytic degradation. Disruption of the secondary and tertiary structure of proteins by oxidative damage has been reported to expose hydrophobic regions of proteins and promote their preferential degradation by proteases. (Cervera, J., Levine, R. L. FASEB J. 2, 2591-2595, 1988; Giulivi, C., et al., Arch. Biochem. Biophys. 311, 329-341, 1994; Pacifici. R. E., et al., J. Biol. Chem. 268, 15405-15411, 1993; Salo, D. C., et al., J. Biol. Chem. 265, 11919-11927, 1990; Davies, K. J. A., et al., J. Biol. Chem. 262, 9914-9920 1987) In many cases, oxidation appears to result in a partial unfolding or rearrangement of target proteins, which exposes the proteolytically susceptible hydrophobic sequences that are normally shielded from enzyme hydrolysis. Any of these changes could also result in potentially irreversible damage of the tissue.

The data of the present invention agree with those of Davies et al.. (1993) (Davies, J. M., et al., Free Radic Biol Med 15(6), 637-43, 1993) that no fragmentation of either collagen takes place in response to physiological concentrations of HOCl (12.5-50 μM). However, at these concentrations HOCl the present invention reveals that HOCl directly oxidizes human collagen type I from bone and collagen type II from articular cartilage. The direct oxidation of collagen types I and II is demonstrated by talking advantage of the fact that N-chloramines. formed by the reaction of HOCl with primary amines, decompose to aldehydes (Thomas, E. L., et al., Methods Enzymol 132, 569-85, 1986; Hazen, S. L., et al., Biochemistry 37, 6864-6873, 1998) and that the formation of N-chloramines contributes to the decrease in silver staining intensity of HOCl-reacted protein. The direct oxidation of Pyd cross-links in collagen type II is also demonstrated by taking advantage of the fact that aldehydes are also formed in the reaction of HOCl with pyridinium compounds, and that the formation of aldehydes results in the disruption of the pyridinium ring and the loss of Pyd fluorescence (325 ex/400 em). The present invention focuses on the oxidation of the Pyd cross-links because their chemical makeup implies that they are reactive with HOCl (Hazen, S. L., et al., J Clin Invest 98(6), 1283-9, 1996) and possibly O₂(¹Δg). (Hormel, S., and Eyre, D Biochim Biophys Acta 1078, 243-250, 1991) Using the decrease in fluorescence emission as a measure for the reaction of HOCl with the Pyd cross-links is supported by the fact that other fluorescent groups present or generated by oxidation of collagen do not fluoresce at 400 nm (325 nm ex) or have increased fluorescence at this wavelength. These groups include tryptophan residues (275 nm ex/334 nm em) which are not present in these collagens, glycation end products (350 nm ex/430 nm em), and bityrosines (325 nm ex/400 nm em), which when formed have an increased fluorescence emission at 400 nm. (Hazen, S. L., et al., J Clin Invest 98(6), 1283-9, 1996; Vissers, M. C., Winterboum, C. C. Arch Biochem Biophys 285(1), 53-9, 1991).

Although both collagen types I and II are oxidized by HOCl, the reactions differ. Oxidized collagen type II shows a greater amount of carbonyl formation and a decrease in fluorescence (325 ex/400 em), as compared with oxidized collagen type I. The increase in carbonyl formation may have resulted from the reaction of HOCl with Pyd cross-links that are more abundant in this collagen type. (Eyre, D. R. Science 207, 1315, 1980). This reaction will also account for, but may not completely explain, the more intense silver staining of collagen type II since a reaction of HOCl with Pyd cross-links will result in a greater amount of carbonyl formation without a corresponding decrease in primary amines. In support of a reaction of HOCl with the Pyd cross-links, a decrease in fluorescence emission at 400 nm (excitation 325 nm) is detected in the collagen type II samples reacted with HOCl. The formation of aldehydes in the reaction of HOCl with pyridinium compounds is rapid and is detected after only 15 min of incubation at 37° C. as opposed to the slower decomposition of N-chloramines to aldehydes, which can take hours.( Weiss, S. J., et al., Science 222(4624), 625-8, 1983).

In summary, activated neutrophils and monocytes are present in early inflammatory lesions and in later focal regions of acute inflammation in arthritic joints (Woolley, D. E., et al., Ann Rhetum Dis. 56(3), 151-61, 1997) and periodontitis. (Fredriksson, M. I., et al., J Periodontal 70(11), 1355-1360, 1999) The presence of activated neutrophils results in the deposition of MPO onto the ECM surface and the production of the reactive oxidant, HOCl. Unlike a reaction with HOCl, the Pyd cross-links need not be on the surface of the fibril to be accessible to attack by Cl₂ or N-chloramines, which are small and less reactive, so they will diffuse from their sites of production, implying that the Pyd cross-links linking collagen type IX-II and II-II in articular cartilage are assessable to reaction during inflammation associated with articular cartilage. However, the Pyd and deoxypyridinoline cross-links of bone collagen type I are probably less susceptible to oxidation because mineralization of bone collagen type I will prevent access of Cl₂ or N-chloramines to the Pyd cross-links in this tissue, and because the lateral packing of the molecules within the fibril and the spacing between collagen molecules is smaller in bone than cartilage.

The in vivo formation of 3-chloro-pyridinium serves as a specific marker for the production of HOCl and the involvement of MPO in inflammation of tissues containing significant quantities of Pyd cross-links. The methods currently employed do not differentiate between cartilage and bone, therefore there is a long felt and unfulfilled need for the ability to diagnose, and monitor (for example, during the course of therapeutic treatment), inflammatory diseases and conditions associated with articular cartilage. The present invention fulfills this need by detecting and measuring chlorinated products that result from the generation of reactive oxygen species (ROS) by neutrophils, as well as monocytes and macrophages, during an inflammatory response. The present invention discloses methods of detecting 3-chloro-pyridinium, as well as other products resulting from the reaction of HOCl on Pyd cross-links (see supra). The presence of these compound/peptides in joint fluid (see Table III supra), serum, and/or urine will serve as a useful clinical marker for inflammation associated with articular cartilage. The methods of detecting these compound/peptides allow for the diagnosis of inflammatory diseases or conditions, including but not limited to rheumatoid arthritis, juvenile rheumatoid arthritis, periodontitis, and early inflammatory lesions, psoriatic arthritis, osteoarthritis (inflammatory osteoarthritis) gout and calcium pyrophosphate deposition disease (pseudogout), lyme arthropathy, arthritis associated with inflammatory bowel disease. reiter syndrome relapsing polychondritis, and joint implant osteoloysis. Further, the methods of the present invention allow for the efficacy of drug therapy to be determined, thereby allowing the physician to monitor the treatment regimen.

What is claimed is:

1. A method of diagnosing an inflammatory disease or condition associated with articular cartilage or bone surfaces in a mammal, comprising:
a) obtaining a patient sample from said mammal; and
b) detecting an amount of a chlorinated compound/peptide(s) in said sample.

2. The method of claim 1 wherein said detecting step further comprises measuring said amount of said chlorinated compound/peptide(s) in said patient sample.

3. The method of claim 1 wherein said chlorinated compound/peptide(s) are at least one of the group of chlorinated forms of pyridinoline cross-links, lysine/hydroxylysine residues, aromatic amino acids, ketoimines and pyrole cross-links.

4. The method of claim 1 wherein said method comprises the immunospecific binding of an antibody to said chlorinated compound/peptide(s) in said patient sample.

5. The method of claim 1 wherein said method comprises detecting said chlorinated compound/peptide(s) in said patient sample by U.V. absorbance or fluorescence.

6. The method of claim 1 wherein said method comprises detecting said chlorinated compound/peptide(s) in said patient sample by mass spectrometry.

7. A method of monitoring a course of therapeutic agent(s) in treating a diseased condition associated with articular cartilage or bone surfaces in a mammal, comprising:
a) obtaining a patient sample from said mammal;
b) detecting an amount of a chlorinated compound/peptide(s); and
c) measuring said amount of said chlorinated compound/ peptide(s).

8. The method of claim 7 wherein said chlorinated compound/peptide(s) are at least one of the group of chlorinated forms of pyridinoline cross-links, lysine/hydroxylysine residues, aromatic amino acids, ketoimines and pyrole cross-links.

9. The method of claim 7 wherein said method comprises the immunospecific binding of an antibody to said chlorinated compound/peptide(s) in said patient sample.

10. The method of claim 7 wherein said method comprises detecting said chlorinated compound/peptide(s) in said patient sample by U.V. absorbance or fluorescence.

11. The method of claim 7 wherein said method comprises detecting said chlorinated compound/peptide(s) in said patient sample by mass spectrometry.

12. A test kit for detecting and/or measuring a chlorinated compound/ peptide(s) in a mammal, comprising, in one or more containers, an antibody immunospecific for said chlorinated compound/peptide(s), said antibody is detectably labeled.

13. A method for preparing a synovial fluid sample for analysis of a biomarker, comprising:
a) incubating said synovial fluid sample with hyaluronidase;
b) digesting said synovial fluid from step a) with trypsin;
c) treating said synovial fluid from step b) with cyanogen bromide; and
d) analyzing said synovial fluid from step c) for a biomarker.

14. The method of claim 13 wherein said biomarker is a chlorinated peptide.
